(19) United States
(12) Patent Application Publication
(10) Pub. No.: US 2007/0015798 A1
(43) Pub. Date: Jan. 18, 2007

(54) Efficient and stereoselective process for large scale synthesis of (3R)-3-(2,3-dihydrobenzofuran-5-yl)-1,2,3,4-tetrahydropyrrolo[3,4-b]quinolin-9-one derivatives

(76) Inventors: Xun Li, New Hope, PA (US); Sebastien Lemaire, Louvain-la-Neuve (BE); Istvan Marko, Grez-Doiceau (BE); Albert Willemsens, Beerse (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/358,282

(22) Filed: Feb. 21, 2006

Related U.S. Application Data
(60) Provisional application No. 60/656,354, filed on Feb. 25, 2005.
(60) Provisional application No. 60/665,954, filed on Mar. 29, 2005.

(51) Int. Cl. A61K 31/435; C07D 215/00

(52) U.S. Cl. 514/340; 546/152

(57) ABSTRACT

This invention relates to an efficient process for large scale stereoselective production of (3R)-3-(2,3-dihydrobenzofuran-5-yl)-1,2,3,4-tetrahydropyrrolo[3,4-b]quinolin-9-ones and key intermediates used for the preparation of benzofuranyl pyrroloquinolones as potent and selective PDE5 inhibitors for the treatment of erectile dysfunction, as well as pharmaceutical compositions and methods of treatment utilizing these compounds.

### CROSS REFERENCE TO RELATED APPLICATIONS

This present application claims benefit of U.S. Provisional Patent Application Ser. No. 60/656354, filed Feb. 25, 2005, and U.S. Provisional Patent Application Ser. No. 60/665954, filed Mar. 29, 2005, which are incorporated herein by reference in their entirety and for all purposes.

### FIELD OF THE INVENTION

This invention relates to an efficient process for large-scale stereoselective production of (3R)-3-(2,3-dihydrobenzofuran-5-yl)-1,2,3,4-tetrahydropyrrolo[3,4-b]quinolin-9-ones and key intermediates used for the preparation of benzofuranyl pyrroloquinolones as potent and selective PDE5 inhibitors for the treatment of erectile dysfunction, as well as pharmaceutical compositions and methods of treatment utilizing these compounds.

### BACKGROUND OF THE INVENTION

In WO 01/87882, published on Nov. 22, 2001, a number of substituted pyrrolopyridinone derivatives that are useful as phosphodiesterase inhibitors. It further discloses a number of ways to prepare the said pyrrolopyridinone derivatives.

Patents and publications in the family of WO 01/87882 include U.S. Pat. No. 6,818,646 and U.S. Pat. No. 6,635,638; United States Patent Application 2005/0113402 (A1), published May 26, 2005; United States Patent Application 2004/0044021 (A1), published Mar. 24, 2004 and United States Patent Application 2002/0010183 (A1), published Jan. 24, 2002 which are incorporated herein by reference in their entirety and for all purposes.

Interesting compounds described therein are, amongst others, compound 65 (referred to as #65, mentioned in example 50A, on page 74 of the mentioned WO 01/87882) and compound 136 (referred to as #136, mentioned in example 92, on page 101 of the mentioned WO 01/87882). An important intermediate in the synthesis of these compounds is the (3R)-3-(2,3-dihydro-benzofuran-5-yl)-1,2,3,4-tetrahydro-pyrrolo[3,4-b]quinolin-9-one compound with formula (A) as shown hereunder.

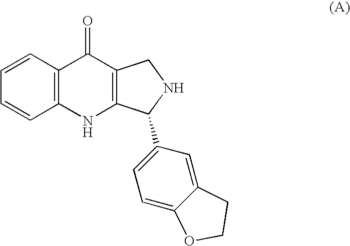

In Tetrahedron Letters 2002, 43, 8941-8945 the synthesis of optically pure pyrroloquinolones via Pictet-Spengler and Winterfeldt reactions was reported. In this article, the catalytic hydrogenation conditions used to remove the chiral auxiliary group produced 5-15% of an over-reduced by-product, and the desired product was 94% e.e., which required an additional chiral HPLC purification to improve the optical purity (≧97% e.e.) of the final product. Therefore, there is a need for an efficient, enantioselective synthesis suitable for the large-scale manufacture of the compounds of formula I.

The present inventive synthesis route is advantageous in a number of ways. The starting materials, tryptamine and 2,3-dihydrobenzo[b]furan-5-carboxaldehyde, are commercially available and inexpensive; and the present route does not require chromatographic purifications to purify intermediates and the final product. Furthermore, the resolving reagent, N-acetyl-D-leucine, used to separate the (R)-enantiomer from the (S)-enantiomer is recovered with high chemical and enantiomeric purity. The (S)-enantiomer enriched mixture is epimerized to a near racemic mixture with high (>93%) yield, which is resolved with the recycled resolving reagent and produced an additional 33±3% yield of the desired (R)-enantiomer with high chemical and enantiomeric purity. All processes are scalable and reproducible.

### SUMMARY OF THE INVENTION

The present invention is directed to a short practical commercial process for the efficient enantioselective synthesis and an efficient production process of a compound of general formula I: 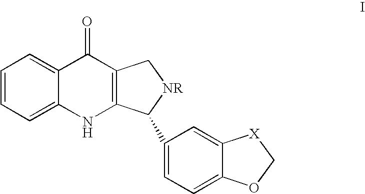
wherein
X is selected from the group consisting of —CH₂—, NH, O and S;
R is selected from H, C₁₋₁₀alkyl, C₁₋₁₀alkyl substituted with C₁₋₆alkyloxy, or C₁₋₁₀alkyl substituted with halo, aryl, heteroaryl, heterocycle, C(O)—C₁₋₁₀alkyl, C(O)-aryl, C(O)O—C₁₋₁₀alkyl or C(O)O-aryl.

The present invention provides a process, which comprises as a first step, the condensation of the readily accessible tryptamine, with an aldehyde of formula II to form a Schiff base of formula XI.

The formation of the Schiff base of formula XI can be carried out in a suitable solvent, such as xylene, toluene or other water-azeotropic solvents.

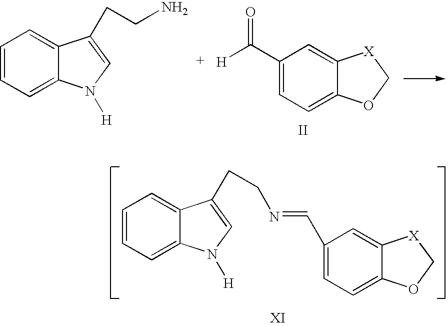

Preferably, the water that is formed during the reaction is removed using a trap such as a Dean-Stark apparatus. When the condensation step is complete, the solvent can be removed, for instance, by evaporation under reduced pressure.

For the subsequent ring closure, the Schiff base of formula XI can be dissolved in a suitable solvent, such as, for example, dichloromethane. An appropriate acid, preferably a strong Brønsted or Lewis acid, for example trifluoroacetic acid, is added as a catalyst.

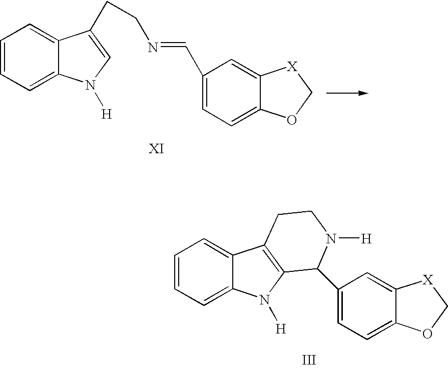

The reaction is performed at a temperature and time sufficient to cyclize to a tetrahydro-β-carboline derivative represented by formula III. Suitable temperatures are dependent upon the solvent used. For example, temperatures of up to 60° C., preferably below 50° C. can be used. After the work-up, the resulting tetrahydro-β-carboline derivative of formula III can be isolated and purified. The reaction can preferably be carried out under an inert atmosphere such as a nitrogen atmosphere. The resulting tetrahydro-β-carboline derivative of formula III is a racemic mixture of (R)-enantiomer of formula XII and (S)-enantiomer of formula XIII.

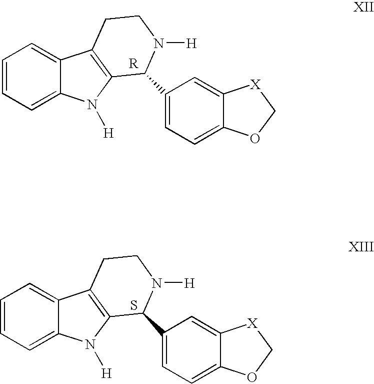

The next step is the reaction of the tetrahydro-β-carboline derivative represented by formula III with a stoichiometric amount of N-acetyl-D-leucine in an organic solvent, preferably a polar solvent, such as for example an alcohol, e.g. methanol. Preferably, the mixture is heated to a temperature and for a period of time that is sufficient to produce the (R)-enantiomer salt of the compound of formula IV with high chemical and enantiomeric purity. The (R)-enantiomer is produced as a crystalline solid, which is essentially free of the (S)-enantiomer. The mother liquor contains a mixture of (R)-enantiomer salt of formula IV and (S)-enantiomer salt of formula VI, which is enriched towards the (S)-enantiomer.

Examples of suitable temperature ranges for the heating are within a temperature range between 30° C. and 80° C. An example of the present invention is a reaction temperature in a range of from about 40° C. to about 70° C. The intermediate of formula III can be added to the reaction mixture, preferably portion wise over a certain period. It was unexpectedly found that the intermediates of formula III could be resolved using N-acetyl-D-leucine. High enantiomeric excesses are obtained by this method.

An example of the present invention is an enantiomeric excess of about 80%. Another example is an enantiomeric excess of about 90%. Another example is an enantiomeric excess of about 95%. Another example is an enantiomeric excess of about 97% or more.

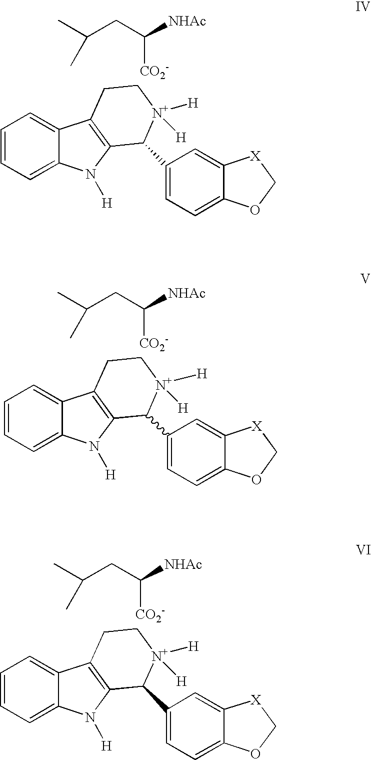

In the case where the reaction does not yield the enantiomeric excess, that was expected, a re-slurry method can be used to obtain purer material.

The resulting mother liquor can be saved for a recycle process. After the isolation of the desired salt, the mother liquor can be concentrated to dryness and the resulting solid material can be dissolved in an appropriate solvent, such as, for example, dichloromethane. An appropriate base can be added, such as, for example an alkali metal hydroxide, e.g. NaOH. The base can be added as a solution in water at a certain concentration (such as for example 1N).

The resulting reaction mixture can be agitated for an appropriate period. Appropriate periods are for example 5 to 30 minutes, preferably about 15 minutes. After the phase separation, the organic phase can be condensed to dryness and can be set aside for epimerization. The aqueous phase can be cooled to a temperature ranging from about 0° C. to about 15° C. and then acidified with an appropriate acid, such as, for example a hydrohalic acid, e.g. a hydrochloric acid. The acid is preferably in a concentrated form so that a pH of about one can be reached. The acid aqueous phase can be then agitated. The addition of concentrated acid is normally an exothermic process and the addition of the concentrated acid can be done slowly.

The resulting solid can be isolated and washed appropriately, such as, for example with water (preferably de-ionized water) at lower temperature. The solid can be dried by, for example, air-suction and is placed in an oven under vacuum in a range of from about 146 to about 210 hPa. The temperature in the drying oven ranges from about 30° C. to about 90° C., preferably around 60° C. for an appropriate period. An appropriate period of time ranges from about 4 hours to about 24 hours. An example of the present invention is a a period of 16 hours.

The resulting solid is the resolving agent N-acetyl-D-leucine in a sufficiently pure form to be used again without further purification. This can be advantageous when applying the present inventive process at larger scales, such as industrial scales.

One of the other inventive aspects of the present process is the epimerization of the intermediate of formula VI to the intermediate of formula V. This epimerization can be very advantageous when applying the process at larger scales, especially industrial scales. When the undesired enantiomer can be converted into the desired enantiomer then that adds to the economic advantage of the process.

The solid, recovered from the organic phase described in the process step to recover the N-acetyl-D-leucine, can be suspended in an appropriate solvent, such as, an apolar solvent, e.g. dichloromethane.

The suspension can be stirred under an inert atmosphere, such as, for example, nitrogen. The suspension can be preferably cooled to a temperature ranging from about 0° C. about 15° C., preferably to a temperature of about 10° C. Then an acid, such as, for example, trifluoroacetic acid can be added to the mixture. The acid can be preferably added over an appropriate period of time and not all at once. After the addition of the acid, the reaction mixture can be heated. The temperature depends on the solvent used.

For example when dichloromethane is used the reaction mixture can be heated to the boiling point of the mixture, i.e. about 40° C., thus allowing the temperature to be maintained under reflux for an appropriate period of time. An appropriate period can be between about 4 hours to about 48 hours, preferably between about 10 to about 24 hours.

An example of the present invention is a period of about 16 hours.

After that, the mixture can be cooled to about 20° C. and preferably can be agitated while an alkaline solution, such as, for example NaOH solution; for instance, a 7% aqueous solution of NaOH can be added over an appropriate period. An appropriate period depends on the actual reactants and the amounts of reactants used. Appropriate periods range from about 5 minutes to 2 hours, preferably about 20 minutes.

Two phases are formed and after phase separation, the organic phase can be washed with brine and subsequently concentrated to dryness. The resulting material can be placed in a vacuum oven under a vacuum, with a pressure in a range of between about 146 hPa to about 210 hPa. The material is a racemic intermediate of formula V and can, as such, be used to undergo resolution again.

The next step in the process can be the protection of the nitrogen atom in the six-membered ring. Several protective groups for the nitrogen atom are available. A number of art-known protective methods are known from the literature, for instance T. W. Greene & P. G. M. Wuts, Protective Groups in Organic Synthesis, 3rd Edition, John Wiley & Sons, 1999.

For instance, the (R)-enantiomer salt of formula IV can be reacted with a stoichiometric excess of a substituted allyl halide, aryl halide or aryl-alkyl halide, or substituted alkyl/aryl carboxylic acid halide, or dialkyl/diaryl dicarbonate in the presence of an inorganic or organic base, such as potassium carbonate or triethylamine.

This reaction can be performed in a solvent for a time and at a temperature sufficient to produce the N-allyl-, or N-aryl-, or N-alkyl/arylcarbonyl, or alkyl/aryl ester of N-carbonic acid of the (R)-enantiomer of the compound of formula VII. Following the above procedure, the (R)-enantiomer of the compound of formula VII is normally in the filtrate, and the potassium salt of the resolving reagent of the compound of formula Xa is found as a solid.

Interestingly a benzyl halide, preferably benzyl bromide, can be used to protect the nitrogen. The reaction temperature range in that case is between 0° C. and 60° C. The reaction is preferably carried out under inert atmosphere, such as, for instance, a permanent nitrogen flow. The reaction work-up can be done according to art-known methods.

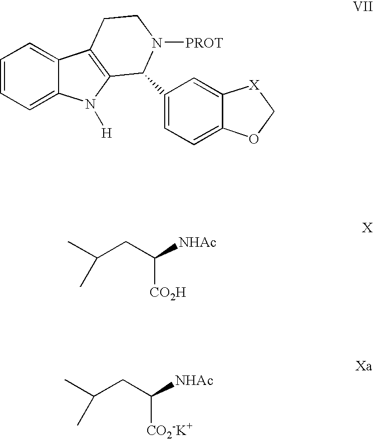

Subsequently the N-acetyl-D-Leucine can be recovered. The purity of the N-acetyl-D-Leucine can be high enough for the N-acetyl-D-Leucine to be reused as a resolving agent.

The next step in the synthesis can be an oxidative rearrangement. In this rearrangement the protected (R)-enantiomer of formula VII is treated with a large stoichiometric excess of an oxidative agent in the presence of a phase transfer catalyst.

The reaction is performed in a solvent, for a time and at a temperature sufficient to produce a N-allyl-, or N-aryl-, or N-alkyl/arylcarbonyl, or alkyl/aryl ester of N-carbonic acid of (R)-enantiomer of the compound of formula VIII.

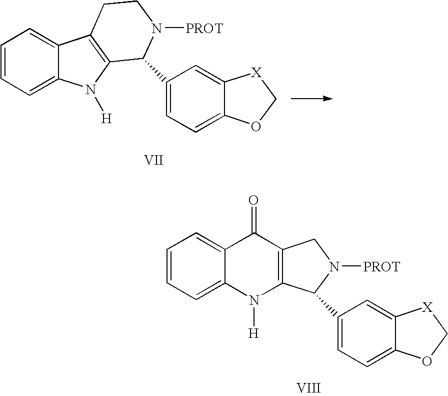

The reaction can be performed by mixing an appropriate oxidant, such as, for example potassium superoxide with an appropriate solvent, such as, dimethylformamide under an inert atmosphere, such as for example, nitrogen, and adding to that mixture an appropriate phase transfer catalyst, such as, for example, Aliquat® 175 (methyltributylammonium chloride).

The reaction mixture can be heated and subsequently the intermediate of formula (VII) dissolved in an appropriate solvent such as dimethylformamide can be added to the mixture. While agitating the resulting reaction mixture the reaction can be kept at a constant temperature between 20° C. to 80° C., preferably between 40° C. and 60° C. for an appropriate period of time which may be a number of hours, for instance 4 hours.

When the reaction has reached its endpoint, the reaction mixture can be cooled and added to water, preferably de-ionized (D.I.) water with fast agitation under nitrogen atmosphere. After stirring for an appropriate time, the reaction mixture can be acidified with a hydrohalic acid, preferably with a hydrochloric acid solution.

The resulting solid can be isolated by filtration and then the filtrate can be washed with an appropriate solvent for instance water. The resultant cake can then be dried by air-suction and placed in a vacuum oven under a vacuum of 146 to 210 hPa at a temperature of about 60° C. for a number of hours, for instance 16 hours.

Further purification can be performed by re-slurrying the resulting solid in an appropriate solvent, such as, for example, an alcohol, e.g. methanol at higher temperatures, such as for instance the boiling point of the alcohol and then cooling down and filtering the resulting mixture.

Subsequently, the intermediate of formula VIII can be de-protected.

The deprotection method depends upon the protecting group that was used.

For instance, the N-allyl-, or N-aryl-, or N-alkyl/arylcarbonyl, or alkyl/aryl ester substituted N-carbonic acid (R)-enantiomer of formula VIII can be reacted under hydrogen pressure in the presence of a stoichiometric amount of an acid in an alcoholic solvent, for a time and temperature sufficient to produce a salt of the (R) enantiomer of formula IX. Suitable catalysts which can be used are platinum or another art-known catalysts, but preferred can be palladium on charcoal. After purging the reactor with nitrogen, the reactor can be purged with hydrogen and then an appropriate pressure of hydrogen can be kept in the reactor (appropriate pressure is in a range of about 3450 kPa).

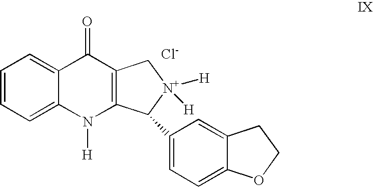

Starting from that deprotected pyrrolidinequinolinone a number of interesting compounds can be made.

### Definitions

The term “C₁₋₁₀alkyl” whether used alone or as part of a substituent group, means a straight or branched chain monovalent hydrocarbon alkyl radical or alkyldiyl linking group comprising from 1 to 10 carbon atoms, wherein the radical is derived by the removal of one hydrogen atom from a single carbon atom and the alkyldiyl linking group is derived by the removal of one hydrogen atom from each of two carbon atoms in the chain, such as, for example methyl, ethyl, 1-propyl, 2-propyl, 1-butyl, 2-butyl, tertiary butyl, 1-pentyl, 2-pentyl, 3-pentyl, 1-hexyl, 2-hexyl, 3-hexyl, 1-heptyl, 2-heptyl, 3-heptyl, 1-octyl, 2-octyl, 3-octyl, 1-nonyl, 2-nonyl, 3-nonyl, 1-decyl, 2-decyl, 3-decyl and the like. Examples include C₁₋₈alkyl, C₁₋₆alkyl and C₁₋₄alkyl groups.

The term “C₁₋₆alkyloxy” means a straight or branched chain alkyloxy group comprising from 1 to 6 carbon atoms, such as, for example, methoxy, ethoxy, propoxy, butoxy, pentoxy, hexoxy and the like. An alkoxy radical may be attached to a core molecule and further substituted where indicated

The term “halo” means includes fluoro, chloro, bromo, and iodo.

The term “aryl,” whether the term is used alone or as part of a substituent group, refers to an aromatic cyclic hydrocarbon ring radical derived by the removal of one hydrogen atom from a single carbon atom of the ring system. Typical aryl radicals include phenyl, naphthalenyl, fluorenyl, indenyl, azulenyl, anthracenyl and the like.

The term “aromatic” refers to a cycloalkylic hydrocarbon ring system having an unsaturated, conjugated π electron system.

The term “heteroaryl,” whether used alone or as part of a substituent group, refers to an heteroaromatic cyclic hydrocarbon ring radical derived by the removal of one hydrogen atom from a single ring carbon atom of the ring system. Typical heteroaryl radicals include furyl, thienyl, pyrrolyl, oxazolyl, thiazolyl, imidazolyl, pyrazolyl, isoxazolyl, isothiazolyl, oxadiazolyl, triazolyl, thiadiazolyl, pyridinyl, pyridazinyl, pyrimidinyl, pyrazinyl, indolizinyl, indolyl, azaindolyl, isoindolyl, benzo[b]furyl, benzo[b]thienyl, indazolyl, azaindazolyl, benzimidazolyl, benzthiazolyl, benzoxazolyl, benzisoxazolyl, benzothiadiazolyl, benzotriazolyl, purinyl, 4H-quinolizinyl, quinolinyl, isoquinolinyl, cinnolinyl, phthalzinyl, quinazolinyl, quinoxalinyl, 1,8-naphthyridinyl, pteridinyl and the like.

The term “heterocycle” whether used alone or as part of a substituent group, refers to a saturated or partially unsaturated monocyclic ring radical derived by the removal of one hydrogen atom from a single carbon or nitrogen ring atom. Typical heterocyclyl radicals include 2H-pyrrole, 2-pyrrolinyl, 3-pyrrolinyl, pyrrolidinyl, 1,3-dioxolanyl, 2-imidazolinyl (also referred to as 4,5-dihydro-1H-imidazolyl), imidazolidinyl, 2-pyrazolinyl, pyrazolidinyl, tetrazolyl, tetrazolidinyl, piperidinyl, 1,4-dioxanyl, morpholinyl, 1,4-dithianyl, thiomorpholinyl, piperazinyl, azetidinyl, azepanyl, hexahydro-1,4-diazepinyl, hexahydro-1,4-oxazepanyl, tetrahydrofuranyl, tetrahydrothienyl, tetrahydropyranyl, tetrahydropyridazinyl, 1,3-benzodioxolyl or 2,3-dihydro-1,4-benzodioxinyl and the like.

The term “high chemical purity” means a chemical purity of more than 90%, more specifically a chemical purity of more than 95% and preferably a chemical purity of 98%, determined by HPLC analysis (area normalization %).

The term “enantiomeric purity” refers to an enantiomeric excess (e.e.) of more than 90%, more specifically an enantiomeric excess of more than 95% and preferably an enantiomeric excess of more than 97% determined by chiral HPLC analysis (area normalization %). The term “enantiomeric excess” is as defined in standard chemical textbooks.

The phrase “essentially free of the (S)-enantiomer” as used herein means that the compound contains less that about 4%, preferably less that about 2% of the (S)-enantiomer.

By the term “a strong Brønsted acid” is meant an organic or inorganic acid which is a proton donor including sulfuric acid, hydrochloric acid, hydrobromic acid, methanesulfonic acid, p-toluenesulfonic acid, substituted benzoic acid, acetic acid, trichloroacetic acid, tribromoacetic acid and trifluoroacetic acid. The use of hydrochloric acid, p-toluenesulfonic acid, or trifluoroacetic acid is preferred. The use of trifluoroacetic acid is more preferred.

By the term “a Lewis acid” is meant any molecular species with a vacant orbital to accept a pair of electrons such as, for example, an aluminum halide (e.g. aluminum chloride, or bromide or iodide), boron halide (e.g. boron trichloride, or tribromide, or trifluoride), antimony halide (e.g. antimony (III)/(V) chloride, or fluoride, or iodide), ferric halide (e.g. ferric (III) chloride, or bromide), tin halide (e.g. tin (IV) chloride, or bromide, or fluoride), and zinc halide (e.g. zinc chloride, or bromide). The use of aluminum chloride, boron trifluoride, tin (IV) chloride or ferric (III) chloride is preferred. The use of aluminum chloride or boron trifluoride is more preferred.

By the term “an organic base” is meant an organic tertiary alkyl/aryl/heterocyclic amine including trimethylamine, triethylamine, tripropylamine, N-substituted piperidine, N-substituted morpholine, substituted pyridine, 1,5-diazabicyclo[4.3.0]non-5-ene (DBN), 1,4-diazabicyclo[2.2.2]octane (Dabco™), 1,8-diazabicyclo[5.4.0]undec-7-ene (DBU), and 1,1,3′,3′-tetramethylguanidine (TMG). The use of triethylamine, N-substituted piperidine, pyridine or DBU is preferred. The use of triethylamine or DBU is more preferred.

By the phrase “substituted allyl or aryl halides” is meant a straight or branched allyl halide and substituted benzyl halides including allyl chloride, allyl bromide, methylallyl chloride, methylallyl bromide, substituted benzyl chloride, benzyl bromide, benzyl iodide. The use of substituted allyl bromide, benzyl chloride or benzyl bromide is preferred. The use of benzyl bromide is more preferred.

By the phrase “substituted alkyl/arylcarboxylic acid halide” is meant a straight or branched C₂-C₆ alkylcarboxylic acid or acid halide and substituted arylcarboxylic acid or acid halide including acetyl chloride or bromide, propionyl chloride or bromide, 2-methylpropionyl chloride or bromide, butyryl chloride, 2-methylbutyryl chloride, 2-ethylbutyryl chloride, 2-methylpentanoyl chloride, 2-ethylpentanoyl chloride, cyclopentanylcarboxylic chloride, hexanoyl chloride, 2-methylhexanonyl chloride, 2-ethylhexanoyl chloride and cyclohexanylcarboxylic chloride. Substituted arylcarboxylic acid halide including substituted substituted furan-2-carboxylic acid or acid chloride, 2-thiophenecarbonyl chloride, nicotinoyl chloride, substituted benzoyl chloride. The use of substituted furan-2-carboxylic acid or acid chloride or 2-thiophenecarboxylic acid chloride is preferred. The use of substituted furan-2-carboxylic acid or acid chloride is more preferred.

By the term “dialkyl/diaryl dicarbonate” is meant a straight or branched dialkyl dicarbonate and substituted diaryl dicarbonate including diallyl dicarbonate, di-tert-dibutyl dicarbonate, and dibenzyl dicarbonate. The use of di-tert-dibutyl dicarbonate or dibenzyl dicarbonate is preferred. The use of dibenzyl dicarbonate is more preferred.

By the term “an oxidative agent” or “oxidant” is meant an inorganic or organic oxygen-containing compound which can cleave a carbon-carbon double bond to two carbonyls, such agents include sodium perchlorate, sodium perborate, sodium periodate, potassium permanganate, sodium permanganate, potassium superoxide (KO₂), sodium persulfate, sodium hypochlorite, hydrogen peroxide, singlet oxygen, oxygen (O₂), ozone, 3-chloroperoxybenzoic acid (mCPBA) or potassium peroxy monosulfate (OXONE®, E.I. Dupont DeMours brand of potassium peroxy monosulfate). An example of the present invention includes the use of reagents such as hydrogen peroxide, mCPBA, OXONE®, potassium superoxide, O₂/NaH or t-BuOK. The use of hydrogen peroxide or potassium superoxide are is preferred.

By the term “a phase transfer catalyst (PTC)” is meant an organic alkyl/aryl quaternary ammonium halide salt including methyltributylammonium chloride (Aliquat® 175), tricaprylylmethylammonium chloride (Aliquat® 366), tetraethylammonium bromide (TEAB), tetrabutylammonium bromide (TBAB), triethylbenzylammonium chloride and 1,4,7,10,13,16-hexaoxacyclooctadencane (18-crown-6). The use of Aliquat®175, Aliquat® 366 or 18-crown-6 is preferred. Use of Aliquat® 175 is more preferred.

### MORE DETAILED DESCRIPTION OF EMBODIMENTS OF THE INVENTION

In Scheme I, an overview is given of a possible process to obtain compounds of formula I. This scheme is shown as an example.

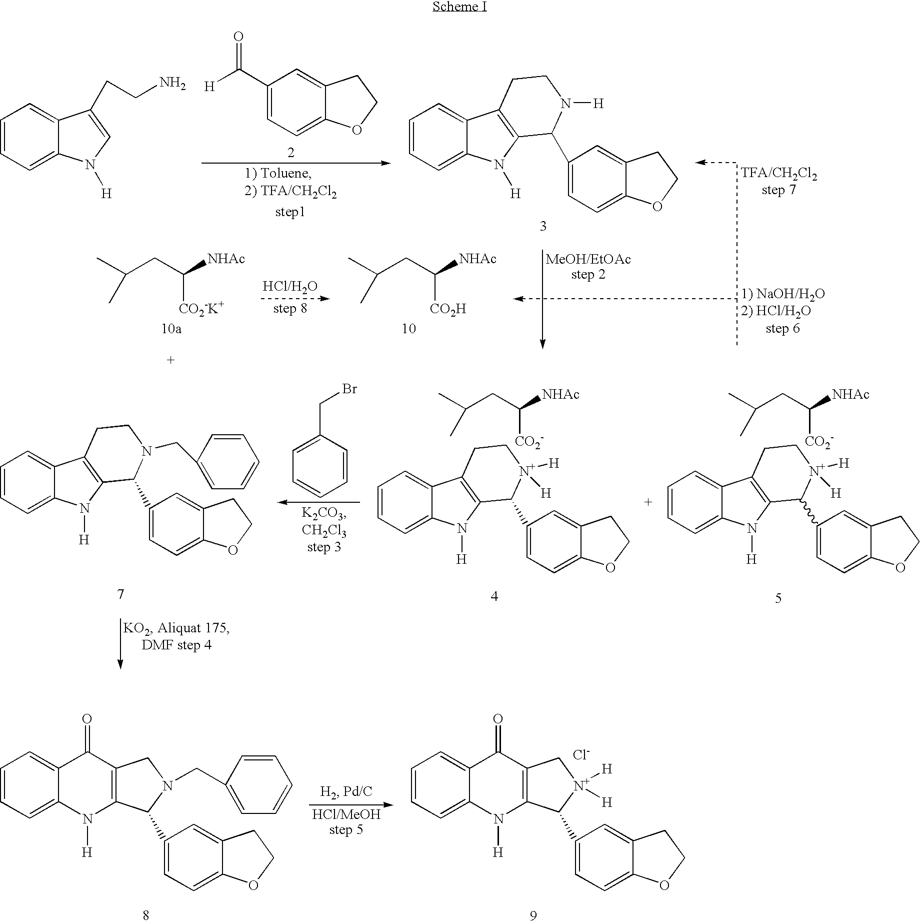

Details of the steps in the Scheme I are provided herein below.

Step 1. Formation of the formula 3 compound, involves a Pictet-Spengler condensation reaction of tryptamine, with the aldehyde of formula 2 in the presence of a solvent to produce a Schiff base in situ. The preferred solvent is toluene, but other aromatic hydrocarbons such as xylene; or an ether such as diglyme (2-methoxyethyl ether) may also be used.

The condensation reaction is normally carried out by heating the reaction mixture of tryptamine and Compound 2 in toluene to reflux temperature under an inert atmosphere such as nitrogen or argon. The reaction is—driven to completion by removal of water from the reaction mixture. Removal of water is achieved by azeotropic distillation of toluene (or other solvent used) and water in the presence of a Dean-Stark trap.

The reaction is refluxed for a sufficient time, preferably at least 6 hours, but as much as 10 hours, to complete the formation of Schiff base. The cyclization of the intermediate Schiff base to the Compound 3 is an acid-catalyzed process and the rate of reaction is dependant upon the concentration, temperature, solvent and acid involved in the reaction. The solubility of the Schiff base is low in aromatic hydrocarbon at room temperature. The complete formation (≧95%) of the Schiff base compound is determined by HPLC and LC-MS analyses. About 80% volume of aromatic hydrocarbon solvent is removed by means of distillation under reduced pressure.

After the reaction mixture is cooled to below 40° C., an equal volume (80%) of chlorinated hydrocarbon such as methylene chloride is added, in which the cyclization is performed in the presence of a stoichiometric excess of an acid. Trifluoroacetic acid (TFA) is preferred, but other strong Brønsted acids such as methanesulfonic acid and p-toluenesulfonic acid, or Lewis acids such as aluminum chloride and boron trifluoride may also be used. The acidic reaction mixture is stirred at room temperature, for a sufficient time preferably at least 16 hours, but as much as 24 hours, to complete the cyclization.

The excess TFA is removed by means of base-washing the reaction mixture with an aqueous alkaline solution to afford the free amine of formula 3. A saturated sodium hydrogen carbonate solution is preferred, but other general alkaline solutions such as sodium carbonate, sodium hydroxide and potassium hydroxide may also be used. The halogenated hydrocarbon solvent is removed (>90% of volume) by means of distillation, and then the crude Compound 3 is dissolved in a solvent mixture of ethyl acetate and hexane (3/5; volume/volume) at about 50±5° C. and the mixture is heated to reflux preferably at least 10 minutes, but as much as 30 minutes, to form a homogenous solution.

The solution is gradually cooled to room temperature over a 1-2 hour period, seeded if necessary, and the resulting slurry is stirred at ice-water temperature for preferably at least 16 hours but as much as 24 hours. The crystalline racemic mixture of the compound of formula 3 is isolated by filtration and dried in a vacuum oven at 55° C. to produce a ≧84% yield (from tryptamine) of racemic Compound 3 which is 97.4% chemically pure.

Step 2. The present invention uses N-acetyl-D-leucine of formula 10 as the resolving agent, to separate the racemic compound of formula 3 into N-acetyl-D-leucine salt of formula 4 in good yield (70% over 3 cycles) and high chemical and optical purity (>97% e.e.). N-Acetyl-D-leucine is prepared by an improved method described in J. Am. Chem. Soc., 1951, 73, 3359-3360.

The present invention is much improved over the prior art process published in Synthesis and Applications of Isotopically Labeled Compounds, Volume 7, 170-173, 2001 (Edited by U. Pleiss and R. Voges, John Wiley & Sons, Ltd.) which describes a two-step method for chemical resolution of another racemic β-carboline, involving (R)-(−)- or (S)-(+)-camphorsulfonic acid in the first step, and then (R,R)-(+)- or (S,S)-(−)-tartaric acid in the second step to produce 29-33% yields of the desired (R)- or (S)-enantiomer of β-carboline salt with high optical purity (>99% d.e.).

The process of the present invention makes use of only one resolving agent, N-acetyl-D-leucine, to produce N-acetyl-D-leucine salt of formula 4 as a crystalline solid that is collected by filtration in 32-35% yield in a single step with high chemical (>97%) and optical purity (>97% e.e.).

The mother liquor is mostly N-acetyl-D-leucine (S)-enantiomer enriched salt mixture of the compound of formula 5, which is recycled to (S)-enantiomer enriched free base mixture (R-/S-enantiomers, mixture ranging from 15%/85% to 35%/65%) in step 6. The (S)-enantiomer enriched free base is further converted to the near racemic mixture of formula 3 in step 7, and the sodium salt N-acetyl-D-leucine is recovered as resolving agent in step 6.

The recycling process produces an additional 38% yield of N-acetyl-D-leucine salt of formula 4 after two more cycles with high chemical and optical purity (>97% e.e.). The process of recycle may be repeated at least three times without new impurities generated. The step 2 of the present invention is characterized by heating a solution of 4.5±0.5 mole of the resolving agent of the compound of formula 10 in about 5 to 9 liters, more preferably 6 to 7 liters of methanol to 50° C. Other solvents such as ethanol, 2-propanol and acetonitrile may be used, but methanol is preferred. One stoichiometric equivalent (4 to 6 moles, more preferable 4.5 to 5.5 moles) of racemic compound of formula 3 is added at a preferable temperature range 45° C. to 65° C., more preferable about 50 to 55° C.

Ethyl acetate (1 to 3 liters, more preferable 1.5 to 2.0 liters) is added slowly, and the formed thick slurry in the mixed methanol/ethyl acetate (4.4/1.0) solvent system is heated to a temperature range 50 to 70° C., more preferable about 62 to 55° C. for 10 to 40 minutes, more preferably 20 to 30 minutes. The reaction mixture is preferably agitated under an inert atmosphere such as nitrogen or argon during the heating and cooling process. The reaction mixture is cooled to 20° C. for about 1 to 10 hours, preferably 2 to 3 hours and agitated for about 16 to 24 hour, preferably 18 to 20 hours; then the optically pure N-acetyl-D-leucine salt of the compound of formula 4 is isolated by filtration and washed with ice-cold methanol and dried.

The use of methanol alone produced a heavy thick slurry which is hard to filter in low yield (21%) but with high optical purity (>97% e.e.), with ethanol or 2-propanol gave low optical pure salt (<90% e.e.), but with ethyl acetate or acetone, no resolution is obtained. When the enantiomeric purity of the isolated salt of formula IV is below 97% e.e., the optical purity is improved by a methanol/ethyl acetate (5.0/1.0) solvent re-slurry process.

Step 3. The protecting reagent used for the N-alkylation of the salt of formula 4 are aryl halides or dialkyl/diaryl dicarbonate, for example, dibenzyl dicarbonate, benzyl chloride or bromide in the presence of potassium carbonate, use of benzyl bromide is more preferred. The presence of the resolving agent of formula 10 does not interfere with the N-benzylation, and no chromatography is required as the resolving agent of formula 10 is converted into the insoluble potassium salt of formula 10a in methylene chloride, which is isolated by filtration and converted to the resolving agent of formula 10 in step 8. The pure N-benzylated compound of formula 7 is obtained in quantitative yield and high optical purity (>97% e.e.) after removal of the solvent by means of concentration.

The step 3 process of the present invention is characterized by 2.5±0.5 moles charge of the salt of formula 4 and 2.2 equivalents of potassium carbonate in methylene chloride at 20° C. and stirred for about 5 to 30 minutes, preferably 10 to 20 minutes. Other inorganic bases such as sodium hydroxide, potassium hydroxide and sodium carbonate may also be used, sodium carbonate and potassium carbonate are more preferred, and the fine powder of potassium carbonate (˜325 mesh, Aldrich) is most preferred. A slightly stoichiometric excess (about 1.0 to 1.5 equivalents, or more preferable about 1.01 to 1.1 equivalents) of benzyl bromide is added over about 10 to 40 minutes, preferably 20 to 25 minutes.

The reaction mixture is preferably agitated under an inert atmosphere such as nitrogen or argon for about 20 to 60 minutes, preferably 30 to 40 minutes at 20° C. The reaction mixture is gently heated between 30° C. to 40° C., preferably between 38° C. to 40° C.; for about 8 to 16 hours, preferably 10 to 12 hours. Then the reaction is cooled to 20° C. and the insoluble white solid is collected by filtration, and the filtration cake is washed with methylene chloride and dried to afford quantitative yield of potassium salt of formula 10a.

The optically pure N-benzylated compound of formula 7 is obtained after removal of the filtrate solvent by means of concentration. N-Benzylation of the formula IV salt in non-chlorinated solvents such as acetone or acetonitrile also afforded a completed reaction, but 3-5% of the potassium salt of formula 10a is soluble in acetone or acetonitrile. Methylene chloride, however, is more preferred in this particular step.

Step 4. The Winterfeldt oxidation prior art (Liebigs Ann. Chem., 1971, 745, 23-30) of the compound in formula 7 uses oxygen in the presence of potassium tert-butoxide (t-BuOK) or sodium hydride (NaH) in dimethylformamide to produce 45-48% of the compound in formula 8 (Tetrahedron Letters 2002, 43, 8941-8945). This yield fell to 20% on scale up and the product required purification by column chromatography.

A modified Winterfeldt oxidation makes use of low cost potassium superoxide (KO₂) in the presence of one equivalent of expensive 1,4,7,10,13,16-hexaoxacyclooctadencane (18-crown-6) as the phase transfer catalyst (PTC) in DMF (Organic Letters, 2003, 5, 43-46). The use of KO₂ as modified herein, compared to. the prior art use of KO₂ described in the original Winterfeldt protocol, is superior for base-sensitive substrates and afford 35-58% yields of the product, which was subsequently purified by column chromatography.

The process of the present invention is characterized by charge of 2 to 5 moles, preferably 3 to 4 moles of KO₂ in DMF (1 to 2 liters) in the presence of less expensive, water-soluble catalyst Aliquat® 175 (methyltributylammonium chloride) (20 mole % relative to the compound of formula VII). Other phase transfer catalysts such as Aliquat® 366 (tricaprylylmethylammonium chloride), tetraethylammonium bromide or triethylbenzylammonium chloride may also be used (Organic Letters, 2003, 5, 43-46).

Aliquat® 175 is the preferred catalyst which catalyzes the reaction to completion within 4 hours, but as much as to 8 hours, and is easy to remove during work up. The solvents such as tetrahydrofuran (THF) or dimethyl sulfoxide (DMSO) may also be used, but DMF is preferred. The reaction mixture is warmed to 30 to 50° C., more preferably 40 to 45° C. for about 20 to 40 minutes, preferably 20 to 25 minutes.

The reaction mixture is preferably agitated under an inert atmosphere such as nitrogen or argon during the heating, cooling or work-up processes. The solution of the compound of formula 7 (0.3 to 1.0 mole, more preferable 0.5 to 0.8 mole) in DMF (0.3 to 1.0 Liter, more preferable 0.4 to 0.6 Liter) is added over 30 to 60 minutes, preferably 30 to 40 minutes. The reaction temperature is maintained between 30° C. to 60° C., preferably 40° C. to 60° C., more preferably 45° C. to 50° C. which is controlled by careful adjustment of the addition rate of the Compound 7 in DMF.

The reaction is initiated at above 45° C. and the latent exothermic reaction could last for 4 to 6 hours when the reaction temperature is maintained at between about 40° C. and 60° C. However, the reaction temperature can reach 115° C. within 3 minutes if the reaction temperature is higher than 60° C. A jacketed vessel connected to a cooling-heating circulator can be used for this reaction.

The reaction mixture is agitated at about 40° C. to 60° C., preferably 45° C. to 50° C. for about 4 to 8 hours, preferably 4 to 6 hours until the data of HPLC analysis determined about 5%, preferably less than 3% of the starting compound of formula 7 in the reaction mixture. The reaction is cooled to 20° C. and the resulting slurry is slowly transferred into another reaction vessel, which is charged with water (4 to 6 liters) and ice (4 to 6 kg) with fast agitation for about 20 to 60 minutes, preferably 30 to 40 minutes.

After the cease of gas (oxygen (O₂)) evolution, the aqueous pH is adjusted to about 7 to 10, preferably to a pH between 8 and 9, with 3N hydrochloride solution. The solid is isolated by filtration, washed with water and dried to afford the crude material of formula 8 in 61% yield. The reaction solution yields is at least 70%, but not more than 85%, (batch to batch variation as determined by ¹H-NMR). Recrystallization of the crude compound of formula 8 from methanol at about 40 to 65° C., preferably 55 to 65° C., afford the pure compound of formula 8 in 42% reaction yield with high chemical (>97%) and optical purity (>97% e.e.). Solvents ethanol, 2-propanol, or a mixture of methanol and water (1/1) may also be used to increase the recovery yield of the compound 8.

Step 5. The prior art process, hydrogenolysis of chiral N-benzyl compound of formula 8 was carried out in ethanol in the presence of 0.20 equivalent of Pd (10% on activated carbon) as catalyst and one equivalent of hydrochloride, under 2413 hPa to 3102 hPa of hydrogen pressure at 25° C. for about 5 hours. The prior art process produced about 5% of over-reduced product and 72% yield of the desired compound of formula A with ≧94% e.e., which required chiral HPLC purification as described as Tetrahedron Letters 2002, 43, page 8943.

The process of the present invention is characterized by charge of 0.6 to 1.2 mole, preferably 0.8 to 1.0 mole of N-benzyl compound of formula 8 in methanol in a floor stand 6-L Parr pressure reactor under an inert atmosphere such as nitrogen or argon at 20° C. with moderate agitation (300 to 500 rpm, 400 rpm is preferred). Solvents ethanol or 2-propanol may be used but methanol is preferred.

A slight excess (1.01 equivalent) of 6N hydrochloride (HCl) solution is added, 1N to 10N of HCl solution may be used, 3N to 8N is preferred, 5N to 6N is more preferred. The catalyst (0.067 equivalent) of Pd (5% on activated carbon) is added, less of catalyst Pd on carbon such as 0.03 to 0.05 equivalent may be used. The reactor is purged with hydrogen (under 1380 hPa) twice.

The reaction mixture is stirred for about 4 to 6 hours, more preferably 4 hours at 20 to 30° C., preferably between 23 to 28° C. under 2758 hPa to 4136 hPa, more preferably 3102 hPa to 3792 hPa, most preferably 3309 hPa to 3947 hPa of hydrogen pressure.

After the reaction is complete, the catalyst is removed by filtration through a Celite® 545 cartridge and washed with methanol. The filtrate is concentrated and dried to afford quantitative yield (99.6%) of formula 9 salt with high chemical (>97%, HPLC area %) and optical purity (>97% e.e; chiral HPLC area %).

Step 6. Both the conversion of (S)-enantiomer enriched salt (R-/S-enantiomers, ranging from a 15%/85% mixture to a 35%/65% mixture of formula 5 to the (S)-enantiomer enriched free base mixture (R-/S-enantiomers, ranging from a 15%/85% mixture to a 35%/65% mixture) in step 6, make the process of the present invention more economical and efficient. (S)-Enantiomer enriched free base mixture (R-/S-enantiomers, ranging from a 15%/85% mixture to a 35%/65% mixture) is further epimerized into a racemic mixture of the compound of formula 3 in step 7, while the potassium salt and sodium salt of resolving agent are recovered as the free acid of formula 10 in this step (as well as in step 8).

After removal of the solvent methanol, the resulting material is dissolved in methylene chloride (6.0 L) and agitated vigorously with 1N to 5 N, preferably 1N to 3N of sodium hydroxide solution 2 to 6 liters, preferably 3 to 4 liters for about 10 to 30 minutes, preferably 15 to 20 minutes. After phase separation, the organic phase is condensed to dryness to afford (S)-enantiomer enriched free base mixture (R-/S-enantiomers, ranging from a 15%/85% mixture to a 35%/65% mixture).

The aqueous phase containing sodium salt of the resolving agent is cooled to 0° C. and the aqueous pH is adjusted to pH=1 with the addition of concentrated hydrochloric acid solution. This is an exothermic process and the solution temperature is maintained between 0° C. to 5° C., preferably 0° C. to 2° C., by controlling the addition rate of acid solution. After the completion of the acid addition, the white slurry is stirred at 0° C. for about 20 to 60 minutes, preferably 30 to 40 minutes. Then the solid is isolated by filtration, washed with cold water and dried to afford the recovered resolving agent of formula 10 in 73 to 80% yield with high chemical (>98%, HPLC area %) and optical purity (>97%, e.e.; chiral HPLC area%).

Step 7. (S)-Enantiomer enriched free base mixture (R-/S-enantiomers in a mixture ratio in a range of about 15%/85% to about 35%/65%) obtained from step 6 is epimerized into a near racemic mixture (R-/S-enantiomers in a mixture ratio in a range of about 44%/56% to about 50%/50%) of formula 3 in step 7.

The process of the present invention is characterized by a charge in a range of from about 2 to about 5 moles, preferably from about 3 to about 4 moles of the compound in a volume of from about 5 to about 8 liters, preferably from about 6 to about 7 liters of methylene chloride. The suspension is stirred under an inert atmosphere such as nitrogen or argon and is cooled to a temperature of from about 0 to about 10° C., preferably of from about 6 to about 10° C. An excess (4 to 8 mole, preferably 6 to 7 mole) of TFA is added over 30 to 60 minutes, preferably 30 to 40 minutes. The addition of TFA is an exothermic process, the suspension becomes a homogenous solution, and the solution temperature is about 25 to 30° C. with cooling after the addition.

The reaction mixture is heated to gentle reflux at about 30 to 40° C., preferably 36 to 38° C. for 12 to 24 hours, preferably 16 to 18 hours. More TFA (0.2 to 0.5 equivalents) can be added and the reaction can be refluxed for an extended time until the epimerization is completed (R-/S-enantiomers, ranging from 44%/56% to 50%/50%, determined by chiral HPLC analysis).

The reaction is cooled to 20° C. and then agitated vigorously while a 5 to 20%, preferably 7 to 10% sodium hydroxide solution is added over 20 to 40 minutes. Other alkaline solutions such as potassium hydroxide, potassium carbonate or sodium carbonate may be used. The organic phase is separated, washed with brine and concentrated to afford 93% of the compound in formula 3 with high chemical purity (>97%, HPLC area %), which is further used in step 3 with the resolving agent of formula 10 to produce more than 36% yield of (R)-enantiomer of formula 4 after two recycles and no new impurities detected.

Step 8. The conversion of the potassium salt 10a generated from step 3 to resolving agent of formula 10 is a similar process as described in step 6 of the present invention.

Step 8 is characterized by dissolving 2 to 5 moles, preferably 3 to 4 moles of potassium salt of formula 10a in 3 to 6 liters, preferably 4 to 5 liters of water and the solution is cooled to about 0 to 10° C., preferably 0 to 4° C. The aqueous pH is adjusted to pH=1 with the addition of concentrated hydrochloric acid solution, while the solution temperature is maintained between 0 to 5° C., preferably 0 to 2° C. by controlling the addition rate of acid solution.

After completion of the addition, the white slurry is stirred at 0° C. for about 20 to 60 minutes, preferably 30 to 40 minutes. Then the solid is isolated by filtration, washed with cold water and dried to afford the recovered resolving agent of formula 10 in 80% yield with high chemical purity (>98%, HPLC area %) and high optical purity (>97%, e.e.; chiral HPLC area %). The potassium salt and sodium salt of formula of N-acetyl-D-leucine may be combined to produce the resolving agent acid of formula 10 as described hereinabove.

### EXPERIMENTAL (EXAMPLES 1-9)

Starting materials, reagents, and solvents were obtained from commercial suppliers and were used without further purification. In general, the water used in the experiments was de-ionized water (D.I. H₂O), some experiments used tap water. The phrase “under house vacuum” is a vacuum pressure of about 146 hPa to about 210 hPa, except where specified. The aqueous pH was measured by pH indicator strips (colorpHast® pH 0-14, EM Science). ¹H NMR spectra were recorded at 300 Hz on a Bruker Avance-300 instrument, while mass spectra were recorded on a Hewlett-Packard Series 1100 LC/MS instrument.

Both chemical and optical purities of most compounds were determined on a HPLC system (Agilent Series 1100) with HPLC Method A or HPLC Method B.

HPLC Method A for chemical purity: ZORBAX Ecilipse XDB-Phenyl column (4.6 mm ID ×150 mm, 3.5 micron) at 40° C. with flow rate of 1.0 mL/min and run time of 10.0 min. UVmax of between 254 and 280 nm. Solvents: Solvent (A) a mixture of 80% H₂O, 0.05% TFA, Solvent (B) 20% MeCN; Gradient (B) 20%/0.0 min, 20%/1.0 min, 90%/6.0 min, 90%/8.0 min, 55%/9.0 min, 20%/10.0 min.

HPLC Method B for optical purity: CHIRALPAK AD-RH (4.6 mm ID×100 mm, 3.5 micron) at 20° C. with flow rate of 1.0 min and with a run time of 20.0 min. UVmax=254 and 280 nm. Solvents: Solvent (A) 90% IPA, Solvent (B) 10% hexane.

The chemical and optical purities of N-acetyl-D-leucine and its salts were determined by HPLC Method C or HPLC Method D.

HPLC Method C for chemical purity: Supelcosil LC-8DB column (4.6 mm ID×150 mm, 5 micron) at 20° C. with flow rate of 1.0 mL/min and run time of 20.0 min. UVmax=210 nm. Solvents: Solvent (A) a mixture of H₂O and 0.05% TFA, Solvent (B) MeCN+0.05%; Gradient (B) 5%/0.0 min, 50%/0-10 min, 50%/10-12 min, 5%/12-13 min, 5%/13-20 min. Retention time 7.0 min/N-acetyl-D-leucine.

HPLC Method D for optical purity: CHIRALPAK AD (4.6 mm ID×250 mm, 3.5 micron) at 20° C. with flow rate of 1.0 mL/min and with a run time of 15.0 min. UVmax=210 nm. Solvents: Solvent (A) 25% IPA, Solvent (B) a mixture of 75% hexane and 0.05% TFA. Retention time: 5.1 min/N-acetyl-D-leucine, 6.0 min/N-acetyl-L-leucine.

### Example 1 (Corresponding to STEP 1 of Scheme 1)

### Preparation of (±)-1-(2,3-dihydrobenzofuran-5-yl)-2,3,4,9-tetrahydro-1H-β-carboline (Compound 3)

Procedure: A 22 Liter 4-neck round bottom flask was equipped with a thermocouple controller, an overhead mechanical agitator, a Dean-Stark trap with a condenser and nitrogen inlet/outlet adapter. The vessel was charged with toluene (10.0 L) and stirred under nitrogen. 2-(1H-indol-3-yl)ethylamine (tryptamine) (98%, 980.8 g, 6.0 mole,) was added, followed by 2,3-dihydrobenzo[b]furan-5-carboxaldehyde (97%, 916.5 g, 6.0 mole). The suspension mixture was heated to 88±2° C., then the temperature was gradually increased to 112±2° C. at a rate of 2° C. per 5 min. The reaction was refluxed for 4 hours and water (108 mL) was collected by the Dean-Stark trap. HPLC and LC-MS were used to monitor the progress of the reaction to >95% completion. The solvent was removed by distillation under reduced pressure and the resulting material was cooled to below 40° C. to produce a thick yellow semi-solid of the Schiff base.

To the above crude Schiff base, was added methylene chloride (9.0 L) with slow agitation; the suspension was cooled to 0° C. in an ice-bath and TFA (99+%, 1368.2 g, 12.0 mol) was added as a small stream over a 40-min period. The ice-bath was removed and the resultant cherry-colored solution was stirred at 20° C. for 16 hours. HPLC and LC-MS were used to monitor the progress of the reaction (≧95% completion with the presence of 2±1% of starting tryptamine). The reaction solution was washed with saturated NaHCO₃ solution (12.0 L) and the aqueous phase (total about 13.6 L) was separated. Caution should be taken when adding the saturated NaHCO₃ solution to the acidic reaction mixture, due to a large amount of gas (carbon dioxide) that was generated. The final aqueous pH was about 8˜9. The organic phase (total 12.5 L) was transferred into another 22 Liter vessel and the solvents were removed by distillation over about 2 hours; the final temperature of the resultant thick syrup was 50° C. Ethyl acetate (EtOAc, 3.0 L) was added to this crude product at 50° C. with mild stirring, followed by the addition of hexane (5.0 L). The mixture was heated to reflux (64±2° C.) for 10 min, gradually cooled to 20° C., and then seeded and stirred at 4° C. for 16 hours. The crystalline solid was collected by filtration, washed with hexane (1.0 L, 0.5 L),. and dried in an oven at 55° C. under house vacuum for 18 hours. There was obtained 1469 g (84% yield, HPLC=97.4%, area %) of the racemic compound of the formula 3, of which the structure was confirmed with ¹H-NMR and LC-MS analyses; which was used in the next step without further purification.

### Example 2 (STEP 2)

### Preparation of (1R)-1-(2,3-dihydrobenzofuran-5-yl)-2,3,4,9-tetrahydro-1H-β-carbol-2-ium (R)-(−)-2-acetylamino-4-methylpentanoate (Compound 4)

Procedure: A 22 Liter 4-neck vessel was equipped with a thermocouple controller, an overhead mechanic stirrer, and a condenser with a nitrogen inlet/outlet adapter. The vessel was charged with methanol (6.6 L) and N-acetyl-D-leucine (97%, 904.3 g, 4.96 mol; the preparation of N-acetyl-D-leucine is described in EXAMPLE 9) with agitation. The resulting clear solution was heated to 50° C. and the racemic Compound 3 (97.4%, 1500 g, 4.96 mol) was added portion-wise over about a 2 min period. The solution turned a cherry color after stirring for another 5 min, then EtOAc (1.5 L) was added over about a 3 min period. Some salt precipitate was formed within the first 3 min and then the solution quickly became a thick slurry. The thick slurry was heated to 62-65° C. and stirred for 20-30 min. The heating. mantle was removed and the mixture was allowed to gradually cool to 20° C. over a about a 2-3 hour period and agitated mildly for an additional 18 hours. A fast cooling and less agitation time resulted in low yield as well as a poor % e.e. The solid was collected by filtration and the filter cake was washed with ice-cold MeOH (0.7 L×3). The filtrate was a mixture of (S)-enantiomer enriched salt (R-/S-enantiomers, mixture ranging from 15%/85% to 35%/65%) of formula 5 and was saved for recycle (see Steps 6 and 7).

The filter cake was dried by air-suction for 1 hour and then placed in an oven under house vacuum at 60° C. for 24 hours. There was obtained 727.0 g (32% yield, 98.0% diastereomeric excess (d.e.)) of the (R) salt of the Compound 4 as an off-white solid, which was used for step 3 without further purification. The structure of the salt 4 was confirmed with ¹H-NMR, LC-MS and chiral HPLC analyses.

When the R/S ratio of the salts is less than 98.5/1.5, a re-slurry method can be use to obtain purer material. For example, 667.0 g of Compound 4 with 96% d.e. was re-slurried in a mixture of MeOH/EtOAc (1.0 L/0.2 L), following the above described resolution process. There was obtained 610.0 g of pure salt 4 with 98% d.e. Several batches can be combined and re-slurried if necessary.

### Example 3 (STEP 3)

### Preparations of (1R)-2-benzyl-1-(2,3-dihydrobenzofuran-5-yl)-2,3,4,9-tetrahydro-1H-β-carboline (Compound 7) and N-acetyl-D-leucine potassium salt (Compound 10a)

Procedure: A 22 Liter 4-neck vessel was equipped with a thermocouple controller, an overhead stirrer, a pressure-equalization dropping funnel, a condenser with a nitrogen inlet/outlet. The vessel was charged with enantiomeric salt Compound 4 (1368 g, 2.95 mol) and CH₂Cl₂ (12.0 L) with stirring under nitrogen. Potassium carbonate (K₂CO₃, 98%, 913.5 g, 6.49 mol, ˜325 mesh, Aldrich) was added over a 3-min period.

After the mixture was stirred at 20° C. for 10 min, benzyl bromide (98%, 520.0 g, 2.98 mol) was added over a 20-min period (Caution should be taken when handling benzyl bromide, a potent lachrymator) and the suspension was agitated at 20° C. for 30 min. The addition of benzyl bromide was mildly exothermic and the final reaction temperature was 28° C. Heating should be applied only after the exothermic reaction has ceased. The reaction mixture was then heated gently to 38±2° C. and continued to be stirred moderately for 10 hours. The progress of the reaction was determined by HPLC, LC-MS and TLC (MeOH/CH₂Cl₂, 10%/90%).

The reaction was cooled to 20° C. and the white solid was isolated by filtration. A filter with a large surface area was used since the potassium salt 10a was a semi-gelatinous material in CH₂Cl₂ and thus the filtration was a slow process. The filtration cake was washed with CH₂Cl₂ (2.0 L×2) and dried to afford the potassium salt 10a for recycle (see EXAMPLE 6).

The combined organic phases were condensed in vacuo and the resulting off-white yellowish solid was dried in a vacuum oven under house vacuum at 60° C. for 16 hours to afford 1054.0 g (94% yield) of the N-benzyl-(R)-enantiomer of Compound 7, which was chemically pure (≧98%) and optically pure (>97% e.e.). The material was free of the potassium salt 10a (≦0.5%) as determined by ¹H-NMR and HPLC. The N-benzyl Compound 7 was used in the next step without further purification. The structure of Compound 7 was confirmed with its ¹H-NMR, LC-MS and chiral HPLC analyses.

### Example 4 (STEP 4)

### Preparation of (3R)-2-benzyl-3-(2,3-dihydrobenzofuran-5-yl)-1,2,3,4-tetrahydropyrrolo[3,4-b]quinolin-9-one (Compound 8)

Procedure: A 5 Liter 4-neck vessel was equipped with a thermocouple controller, an overhead stirrer, a pressure-equalization dropping funnel, and a condenser with a nitrogen inlet/outlet adapter. The vessel was charged with dimethylformamide (1.5 L) and potassium superoxide (96%, 255.6 g, 3.6 mol) with agitation under nitrogen, followed by Aliquat® 175 (methyltributylammonium chloride, 75 wt. % in H₂O, 39.1 mL, 0.12 mol). The mixture was warmed to 40° C. for 20 min with agitation. Agitation was needed due to the presence of H₂O (˜0.5% of the total volume) from Aliquat® 175. The reaction was quenched by KO₂ before the addition of Compound 7.

A solution (prepared at 50° C.) of Compound 7 (228.0 g, 0.60 mol) in dimethylformamide (0.5 L) was added over a 30 min period while the reaction temperature was maintained between 40 to 60° C. The reaction was initiated at above 45° C.; as a result, the latent exotherm would last for 4 to 6 hours when the reaction temperature was maintained between 40° C. to 60° C. However, the temperature could reach 115° C. within 3 min, if the reaction temperature was higher than 60° C.

After the addition was complete, the heating mantle was removed and replaced with a water bath, and the reaction was agitated at 40 to 60° C. for 4 hours. The progress of the reaction was determined by HPLC, LC-MS and TLC (MeOH/CH₂Cl₂, 5%/95%). A longer reaction time (up to 6 hours) may be needed if there is more than 3 to 5% of the starting Compound 7 present.

The reaction was cooled to 20° C. using an ice-water bath, and the resulting orange slurry was slowly transferred into a 22 Liter 3-neck vessel, which was charged with deionized (D.I.) H₂O (3.6 L) and ice (3.6 kg) with fast agitation under a nitrogen atmosphere. There was a large amount of gas (oxygen) released from the system, therefore, nitrogen blowing directly over the reaction surface helped to reduce the foaming.

The yellowish slurry was stirred for 30 min and acidified with 3 N HCl solution (0.7 L) to a pH of about 8˜9. The solid was isolated by filtration and washed with H₂O (0.5 L×2). The wet filter cake was dried by air-suction and then placed in an oven under house vacuum at 60° C. for 16 hours.

There was obtained 145.0 g (61% yield) of crude Compound 8, which was ground to a fine powder and then transferred to a 3 Liter 3-neck vessel equipped with a thermocouple controller, a mechanical overhead stirrer, a condenser and a nitrogen outlet adapter. The vessel was charged with CH₃OH (0.65 L) and warmed to 64±1° C. for 20 min.

The resultant thick slurry was cooled to 20° C. over a 1 to 2 hours period and then placed in a cold room (about 4° C.) for 16 hours. The solid was isolated by filtration and washed with cold CH₃OH (0.08 L×3 or until colorless). The wet cake was dried by air-suction and then placed in a vacuum oven under house vacuum at 60° C. for 16 hours to afford 99.8 g of 1-(R)-N-benzyl-enantiomer of Compound 8 with high chemical (>97%) and optical purity (>97% e.e.). The structure of Compound 8 was confirmed by ¹H-NMR and LC-MS analyses and was used for next step without further purification.

### Example 5 (STEP 5)

### Preparation of (3R)-3-(2,3-dihydrobenzofuran-5-yl)-9-oxo-2,3,4,9-tetrahydro-1H- pyrrolo[3,4-b]quinoline hydrochloride (Compound 9)

Procedure: A floor stand 6 Liter Parr pressure reactor was equipped with a thermocouple controller, a mechanic overhead stirrer, a cooling coil, nitrogen/hydrogen/vacuum lines and an emergency relief disc. This vessel was charged with Compound 8 (319.2 g, 0.81 mol) and CH₃OH (1.8 L) with fast agitation due to the thick slurry that was formed under nitrogen. A solution of 6 N hydrochloride (0.136 L) was added over 3 min. (this addition was an exothermic process and the temperature of the resulting clean homogenous solution was about 29° C.) followed by the addition of 5% Pd/C (115.2 g, 6.6% mol of Pd) over 1 min. The system was closed and first purged with nitrogen (1380 hPa) (×3) and then hydrogen (1380 hPa) (×2).

The reaction was carried out at 23-28° C. under hydrogen pressure (3447 hPa.) with moderate agitation (400 rpm) for 4 hours. The progress of the reaction was determined by HPLC, LC-MS and TLC (MeOH/CH₂Cl₂, 1/9). The reaction mixture was transferred to a filtration bottle by vacuum, the catalyst was removed by filtration through a Celite® 545 short cartridge, and the filter cake was washed with CH₃OH (0.2 L×4). The Pd catalyst is an air-sensitive flammable metal, thus the surface of the filter cake should never be allowed to become dry.

The filtrate was condensed in vacuo, and the resulting crude material was placed in an oven under house vacuum at 60° C. for 24 hours. There was obtained 275.4 g (99.8%) of quinolinopyrrolidine HCl salt of Compound 9 as a greenish solid. The structure of pyrrolidinequinolinone HCl salt 9 was confirmed with ¹H-NMR and LC-MS analyses. Compound 9 was chemically pure (≧97%) and optically pure (>97% e.e.) and was used in subsequent steps without further purification.

### Example 6 (STEP 6)

### Preparations of (S)-enantiomer enriched 1-(2,3-dihydrobenzofuran-5-yl)-2,3,4,9-tetrahydro-1H-β-carboline free base mixture and N-acetyl-D-leucine sodium salt

Procedure: The filtrate containing the mixture of (S)-enantiomer enriched salt (R-/S-enantiomers, 15%/85% to 35%/65%) of formula 5 from EXAMPLE 2 was concentrated, and the resulting material was dissolved in CH₂Cl₂ (6.0 L) and transferred to a 12 Liter 3-neck separatory flask equipped with an overhead mechanic stirrer. The organic solution was agitated vigorously with a solution of 1 N sodium hydroxide (3.0 L) for at least 15 min.

After the phase separation, the organic phase was condensed to dryness to afford the (S)-enantiomer enriched free base mixture (R-/S-enantiomers, 15%/85% to 35%/65 %), which was used for the epimerization step (see EXAMPLE 7). The aqueous phase (6.0 L) was cooled in an ice bath and acidified with concentrated HCl solution (37%, 1.3 L) to a pH of about 1 with fast agitation. The addition of HCl solution should be slow since this addition was an exothermic process.

The resultant white slurry was stirred at 0° C. for 30 minutes, and the white solid was isolated by filtration and washed with cold D.I. H₂O (0.2 L×2). The solid was dried by air-suction and then placed in a vacuum oven under house vacuum at 60° C. for 16 hours. There was obtained 628.0 g (73% yield) of recovered N-acetyl-D-leucine as a white solid with high chemical purity (>97%, HPLC area %) and optical purity (97% e.e.). The structure was also confirmed by its ¹H-NMR and LC-MS analyses.

### Example 7 (STEP 7)

The present invention is further directed to a process for epimerization of a (S)-enantiomer enriched (1S)-1-(2,3-dihydrobenzofuran-5-yl)-2,3,4,9-tetrahydro-1H-β-carboline mixture (R-/S-enantiomers, mixture ranging from 15%/85% to 35%/65%) to a near racemic (±)-1-(2,3-dihydrobenzofuran-5-yl)-2,3,4,9-tetrahydro-1H-β-carboline mixture (R-/S-enantiomers, mixtures ranging from 44%/56% to 50%/50%)

Procedure: A 12 Liter 4-neck vessel was equipped with a thermocouple controller, an overhead stirrer, a pressure-equalization dropping funnel and a condenser with a nitrogen inlet/outlet adapter. The vessel was charged with the (S)-enantiomer enriched free base mixture (R-/S-enantiomers, mixtures ranging from 15%/85% to 35%/65%) recovered from EXAMPLE 6 (1572 g, 3.39 mol) and CH₂Cl₂ (6.7 L).

The suspension was stirred and cooled in an ice bath to 10° C.; TFA (99+%, 684.0 g, 6.0 mol) was added drop-wise over a 30-min period. The addition was an exothermic process and the temperature of the solution was about 30° C. after the addition, while the heterogeneous mixture became a homogenous solution. The reaction mixture was warmed to gentle reflux at 38±2° C. for 16 hours. The progress of the reaction was determined by chiral HPLC analysis.

The final R-/S-ratio of the racemized mixture was in the range of 44%/56% to 50%/50%. More TFA (0.2-0.5 equivalent) was added and the reaction mixture was refluxed for an extended time. The solution was cooled to 20° C. and agitated vigorously while a 7% NaOH solution (4.3 L, 7.5 mol) was added over a 20 min period. After phase separation, the organic phase was washed with brine (2.0 L) and concentrated to dryness. The resulting material was placed in an oven under house vacuum at 60° C. for 16 hours.

There was recovered 912.3 g (93% yield) of Compound 3 as a yellowish solid with high chemical purity (>97%). The structure of the near racemic Compound 3 was confirmed by ¹H-NMR and LC-MS analyses. Without further purification, the recovered compound was resolved with the recovered N-acetyl-D-leucine Compound 10 (recovered from either EXAMPLE 6 or EXAMPLE 8), to produce an additional 36% yield of the desired 1-(R) enantiomerically pure salt of Compound 4 with high optical purity (≧97% e.e.). The structure of Compound 4 was confirmed with ¹H-NMR, LC-MS and chiral HPLC analyses.

### Example 8 (STEP 8)

### Conversion of N-acetyl-D-leucine potassium salt (Compound 10a) to N-acetyl-D-leucine (Compound 10)

Procedure: A 12 Liter 4-neck vessel was equipped with an overhead mechanical stirrer and a dropping funnel. The vessel was charged with N-acetyl-D-leucine potassium salt (2.95 mol, Compound 10a) (from EXAMPLE 3) and D.I H₂O (3.5 L). The solution was cooled to 4° C. with an ice bath and then a 37% HCl solution was added (1.5 L) over a 30-min period as a large amount of gas (carbon dioxide) was released. The agitation was continued at a temperature of about 0° C. to about 4° C. for an additional 30 min. The pH of the final solution was about pH 1.

The white solid was isolated by filtration, washed with cold D.I. H2O (1 L×2), dried by air-suction and then placed in a vacuum oven under house vacuum at 60° C. for 16 hours. There was obtained 410.0 g (80% yield) of recovered N-acetyl-D-leucine 10 as an off-white solid with a high optical purity (>97% e.e.). The structure of the recovered resolving agent 10 was also confirmed by ¹H-NMR and LC-MS analyses.

### Example 9

### Preparation of (R)-(−)-2-acetylamino-4-methylpentanoic acid (N-acetyl-D-Leucine, Compound 10)

Procedure: The preparation of N-acetyl-D-Leucine of Compound 10 was carried out by a modified prior art process (J. Am. Chem. Soc., 1951, 73, 3359-3360) as shown in Scheme II below and as described herein.

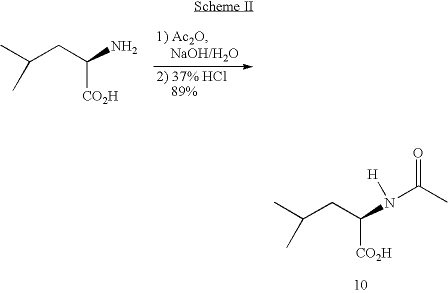

A 22 Liter 4-neck vessel was equipped with a thermocouple controller, an overhead mechanic stirrer and two 2.0 Liter addition funnels. The vessel was charged with D.I. H₂O (2.45 L) and D-leucine (99% e.e., 917.0 g, 7.0 mol) with agitation. Acetic anhydride (99%, 2142.0 g, 21.0 mol) and a 20 N solution of NaOH in H₂O (2.45 L, 49.0 mol) were added simultaneously over a 3 to 4 hour period, while the reaction temperature was maintained between 5 to 15° C. The addition rates of the alkaline solution and acetic anhydride were adjusted along with wet ice cooling to maintain the reaction temperature. The pH of the reaction mixture was maintained slightly alkaline (pH 8˜9) and measured every 10 to 15 min with pH indicator strips. The progress of the reaction was determined by HPLC and LC-MS. After the addition was completed, the mixture was agitated for 1 hour and then acidified cautiously with a 37% HCl solution (4.76 L, 49.0 mol) over a 30-min period. A white solid precipitated and the slurry was stirred for 2 hours at about 5° C. to about 15° C. The resulting solid was isolated by filtration, washed with D.I. H₂O (2.0 L×7), dried by air-suction for 3 hours and then placed in a vacuum oven under house vacuum at 60° C. for 16 hours (drying was stopped when the solid lost no more weight). There was obtained 1083 g (89%) of N-acetyl-D-leucine (Compound 10) as a white powdery solid with high optical purity (>97% e.e.). The structure of Compound 10 was confirmed by ¹H-NMR and LC-MS analyses, and was used in the next step without further purification.

### EXPERIMENTAL (EXAMPLES 10-12)

The present invention is further directed to a synthesis process to provide scalable quantities of (3R)-3-(2,3-dihydro-benzofuran-5-yl)-2-pyridin-2-yl-1,2,3,4-tetrahydro-pyrrolo[3,4-b]quinolin-9-one methanesulfonic acid salt Compound 12 as shown below in Scheme III. More particularly, the present invention is directed to the large scale Buchwald-Hartwig coupling of 2-bromopyridine and a pyrroloquinolone compound of formula (A) to provide an intermediate Compound 11 (represented in WO 01/87882 as Compound 136). The intermediate Compound 11 is then carried forward as described herein to provide Compound 12.

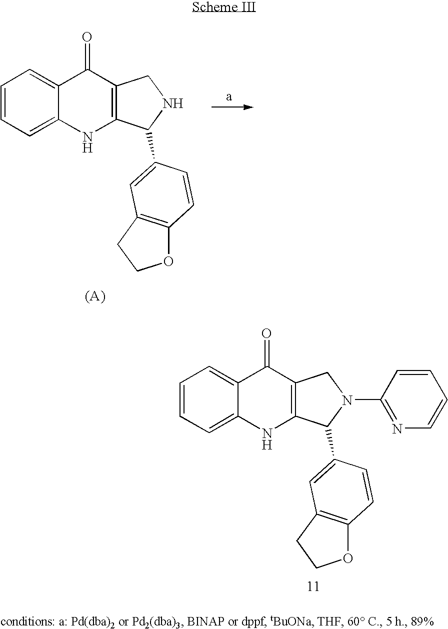

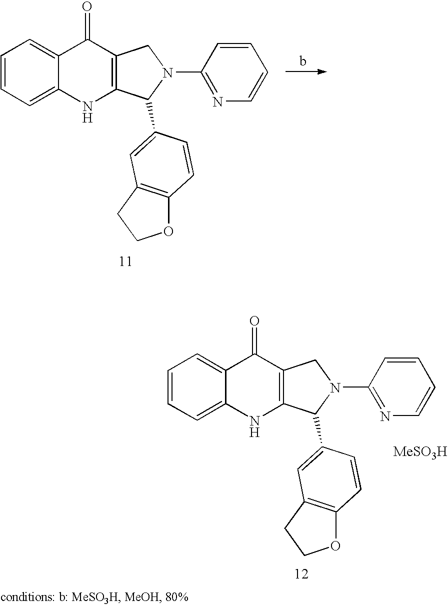

A challenge in the synthesis of (3R)-3-(2,3-dihydro-benzofuran-5-yl)-2-pyridin-2-yl-1,2,3,4-tetrahydro-pyrrolo[3,4-b]quinolin-9-one Compound 11 was the coupling between 2-bromopyridine and the pyrroloquinolone derivative (3R)-3-(2,3-dihydro-benzofuran-5-yl)-1,2,3,4-tetrahydro-pyrrolo[3,4-b]quinolin-9-one compound of formula (A). As represented in Table 1 various conditions were applied:

TABLE 1
Conditions for the Buchwald-Hartwig coupling.

| Entry | Catalyst (mol %) | Ligand (mol %) | Solvent | T (° C.) | Time | Yield |
|---|---|---|---|---|---|---|
| 1 | Pd₂dba₃ (28) | (±)-BINAP (85) | 1,4-dioxane | 90 | 1 h | 30-55%^{a} |
| 2 | Pddba₂ (4) | (±)-BINAP (6) | 1,4-dioxane | 40 | 24 h | 20% |
| 3 | Pddba₂ (4) | (±)-BINAP (6) | THF^{b} | 66 | 16 h | 14-64%^{c} |
| 4 | Pddba₂ (4) | (±)-BINAP (6) | THF^{b,d} | 60 | 10 h | 80% |
| 5 | Pddba₂ (4) | (±)-BINAP (6) | THF^{b,e} | 60 | 9 h | 90% |
| 6 | Pd₂dba₃ (4) | (±)-BINAP (9) | THF^{b,e} | 60 | 5 h | 89% |
| 7 | Pddba₂ (4) | (±)-BINAP (6) | DMF^{b} | 60 | 24 h | <10% |
| 8 | Pddba₂ (4) | (±)-BINAP (6) | DME^{b} | 60 | 24 h | 30% |
| 9 | Pddba₂ (4) | dppf (6) | THF^{b,e} | 60 | 9 h | 80% |
| 10 | Pddba₂ (4) | 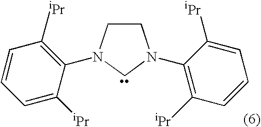 | THF^{b,d} | 60 | 24 h | <5% |
| 11 | Pddba₂ (4) | 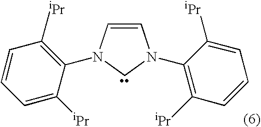 | THF^{b,d} | 60 | 24 h | <5% |

^{a}76% e.e.;
^{b}THF distilled from sodium/benzophenone or extra dry THF, 4Å molecular sieve, (water <50 ppm, stab. from Acros);
^{c}97% e.e.;
^{d}degassed solvent;
^{e}amount of water from 0.01 to 0.1% w/w.

Entry 1

Initial attempts resulted in variable yields (Yield: 30-55%). After 1 hour at 90° C. in 1,4-dioxane, the enantiomeric excess had dropped from >98% to 76%. In order to limit the racemisation, the coupling had to be performed at lower temperature, either in 1,4-dioxane or in THF.

Entry 2

When the reaction was performed at 40° C., after 24 hours, no racemisation could be detected. However, the conversion decreased significantly and the desired product was isolated in only 20% yield.

Entry 3

An improvement was obtained by performing the coupling between 60-66° C. in THF for about 16 hours. Racemisation was barely detectable, but the yields proved to be irreproducible. The best ligand for this reaction was found to be (±)-BINAP.

Entry 4

At a temperature regime of 60-66° C. and a catalyst loading reduced to 4 mol %, a further improvement was observed when the solvent was degassed beforehand. A promising yield of 80% was reached after 10 hours reaction. Based upon this result, all subsequent reactions were performed at 60° C., in the presence of two equivalents of t-BuONa and using 1.2 equivalents of 2-bromopyridine. Additionally, the in situ formation of the catalyst led to more reproducible yields. Therefore, the palladium source and the ligand were mixed together in degassed THF for 30 minutes, then 2-bromopyridine, pyrroloquinolone compound of formula (A) and t-BuONa were sequentially added. The THF used had a water content in a range of from about 0.01% w/w to about 0.1% w/w and led to the same rate and yields as the product dried over sodium/benzophenone. The best ligand for this reaction was found to be (±)-BINAP.

Entry 5

The THF used had a water content in a range of from 0.01% w/w to 0.1% w/w) and led to the same rate and yields as the product dried over sodium/benzophenone. The best ligand for this reaction was found to be (±)-BINAP.

Entry 6

The best ligand for this reaction was found to be (±)-BINAP.

Entry 7

A limited solvent screening showed that THF was the best solvent for the reaction under the indicated conditions. Comparatively, the use of DMF led to lower conversions, presumably due to competitive displacement of the ligand on the palladium catalyst by the solvent, resulting in lower catalytic activity. The best ligand for this reaction was found to be (±)-BINAP.

Entry 8

Again, a limited solvent screening showed that THF was the best solvent for the reaction under the indicated conditions. Comparatively, a poor yield was observed using DME due to the insolubility of the starting material, even at 60° C. Toluene, a popular solvent in Buchwald-Hartwig couplings, proved to be equally unsuitable, due to the poor solubility of the substrate (not shown in Table 1). The best ligand for this reaction was found to be dppf.

Entry 9 and 10

The diaminocarbene palladium complexes exhibited lower activity.

Degassed THF Preparation

A sufficient degassing effect was obtained by refluxing the THF for 30 minutes, followed by nitrogen blanketing.

Salt Crystallization

After work-up, a crude pyrroloquinolone compound 11 was isolated in 89% yield. The methanesulfonic acid salt compound 12 was crystallized from compound 11 via methanol to afford the desired product in 80% yield, with 99% purity and in excess of 99.5% e.e. as the (R)-enantiomer.

The best results were obtained by performing the crystallisation over 20 hours. Increasing the crystallisation time to 72 hours led to reduced yields (70%).

After the crystallization stage, HPLC analysis of the mother liquor revealed a substantial amount (15%) of partially racemised product (74% e.e.), indicating that the final compound was sensitive to acidic conditions. In the solid state, however, no racemisation of the isolated salt was observed.

### Example 10

Preparation of (3R)-3-(2,3-dihydro-benzofuran-5-yl)-2-pyridin-2-yl-1,2,3,4-tetrahydro-pyrrolo[3,4-b]quinolin-9-one (Compound 11)

Procedure: The preparation of Compound 11 is shown in Scheme III and is described herein.

The elution gradient was 5% acetonitrile/95% NH₄OAc (0.5% in water) ramping to 95% acetonitrile/5% NH₄OAc (0.5% in water) over 16 min, at a flow rate of 1.2 mL/min, then kept 95% acetonitrile/5% NH₄OAc (0.5% in water) for 3 min.

Optical purities were measured by Capillary Electrophoresis employing a 57 cm uncoated fused silica column (75 μm I.D., 375 μm O.D., UV wavelength of 200 nm) at 20° C. The mobile phase consisted of a 50 mM phosphate buffer, at pH 3.0; the chiral selector of 10 mM DM-beta-CD. Each run lasted 30 min.

In a 50 Liter glass-lined reactor, was added 25 L of THF and the solvent was heated at reflux (66° C., 90 rpm). After 30 min, the reactor was flushed with a nitrogen atmosphere and cooled to 20° C. Then, 91.57 g of Pd₂dba₃ (0.1 mol, 0.04 mol/mol) was added followed by the addition of 140.1 g of (±)-BINAP (0.225 mol, 0.09 mol/mol). After stirring for 30 min., 474 g of 2-bromopyridine (3.0 mol, 1.2 mol/mol) was added over 10 min. Finally, 815 g of pyrroloquinolone compound of formula (A) (2.5 mol) and 600.6 g of sodium tert-butoxide (6.25 mol, 2.5 mol/mol) were added and the mixture was allowed to warm up to 60° C. and kept at that temperature for 5 hours.

After cooling to room temperature, 50 g of dicalite were added and the mixture was filtered. The cake was washed with 5 L of THF. Then, 20 L of water were added to the organic mixture, followed by 685 mL of aqueous HCl (34.5% w/w). After stirring for 30 min. at room temperature, 10.75 L of ethyl acetate was added and the mixture was stirred for another 15 min. The two layers were separated and the organic layer was discarded. Then, 13.5 L of ethyl acetate was added to the aqueous layer, followed by the addition of 650 mL of aqueous ammonia.

After stirring for 30 min., the aqueous layer was removed and the organic phase was evaporated to dryness (10 L). Compound 11 (1.287 kg, yield 135%) was obtained with a quality of 66.1%, corresponding to an active yield of 89.2%. The enantiomeric excess of the (R)-enantiomer was 99%. The product contained 2530 ppm of residual Pd.

### Example 11

Preparation of (3R)-3-(2,3-dihydro-benzofuran-5-yl)-2-pyridin-2-yl-1,2,3,4-tetrahydro-pyrrolo[3,4-b]quinolin-9-one methanesulfonate salt compound 12

Procedure: The preparation of compound 12 is shown in Scheme III.

A 500 Liter glass-lined reactor was charged with 1.6 kg of compound 11 (3.04 mol, 2110 ppm Pd) and 288.9 L of methanol. After heating to 60° C., the mixture was stirred (75 rpm) for 1 hour at this temperature. Then, 106 g of Norit A Supra and 10.6 g of dicalite were added. The mixture was stirred for another 20 min. and thereafter filtered. The cake was washed with 5 L of methanol. The solvent was reduced to 110 L, at 50° C., and then transferred to a 100 Liter glass-lined reactor where the mixture volume was reduced to 35 L, at 60° C.

Subsequently, methane sulfonic acid (298 g), dissolved in 1 L of methanol, was added over 10 min. to the mixture. Finally, the solution was cooled down to 20° C. and stirred for 20 hours. The mixture was filtered and the cake was washed with 1.5 L of methanol. After drying for 45 hours, 1.17 kg of product compound 12 was obtained (Yield: 80.5%) with a HPLC quality of 100% and >99.5%ee. The amount of residual Pd was below 10 ppm.

Structural data: J. Med. Chem. 2005, 48, 2126-2133; PCT Application WO 01/087882. ¹H NMR (400 MHz, CD₃OD): δ 8.30 (d, 1H, J=9.3 Hz), 8.02 (m, 1H), 7.35 (m, 4H), 7.10 (m, 3H); 6.55 (m, 2H); 4.85 (d, 1H, J=22 Hz), 4.54 (d, 1H , J=22 Hz), 4.40 (t, 2H, J=9.5 Hz); 2.92 (t, 2H, J=9.5 Hz).

### EXPERIMENTAL (EXAMPLES 12-15)

The present invention is further directed to a process for the oxidative rearrangement of the β-carboline derivatives of Formula VII to the quinolone derivatives of Formula VIII. The rearrangement can be performed on an arylated β-carboline derivative of Formula VII (which can yield the final compound directly). Alternatively, the oxidative rearrangement may be performed on a benzyl-protected compound of Formula VII. The resulting quinolone can then be deprotected and subsequently arylated.

The oxidative rearrangement is preferably performed on a protected β-carboline, wherein the protecting group is selected from benzyl, tert-butoxycarbonyl or benzyloxycarbonyl.

Winterfeldt oxidation of 1,2,3,4 tetrahydro-β-carbolines is well known (Boch, M.; Korth, T.; Nelke, J. M.; Pike, D.; Radunz, H.; Winterfeldt, E.; Chem. Ber. 1972, 105, 2126-2142.). With acyl-β-carbolines, however, Winterfeldt oxidation fails to give the desired quinolones (Jiang, W.; Zhang, X.; Sui, Z.; Organic Letters, 2003, 5, 43-46.). The process described herein provides a more efficient and applicable method to oxidize 1,2,3,4 tetrahydro-β-carbolines than is known in the art.

Oxidative cleavage of the 2,3 bond of indoles with KO₂ in presence of a phase transfer catalyst is well described in literature (Baloch-Hergovich, E.; Spier, G.; Tetrahedron Lett. 1982, 23, 4473-4475. Itakura, K.; Uchida, K.; Kawakishi, S.; Tetrahedron Lett. 1992, 33, 2567-2570.).

### Example 12

When referred to as such, the ARC, RC₁ and Phi-tec adiabatic calorimetry experiments were conducted using an ARC calorimeter, manufactured by CSI Columbia Scientific Ind., a RC₁ calorimeter, manufactured by Mettler Toledo and a Phi-tec calorimeter, Model No. Phi-tec II, manufactured by Hel.

KO₂ (8 equivalents) and Et₄NBr (8 equivalents) in DMF were heated to 55° C., then the N-benzyl protected β-carboline of Formula VII was added over 20 minutes. The reaction mixture was stirred for 3 hours to provide the N-benzyl protected quinolone of Formula VIII (20-25% yield).

The reaction mixture had shown a thermal instability at relatively low temperatures. An experiment in a ARC calorimeter on the N-benzyl protected β-carboline of Formula VII reaction mixture showed an exothermic decomposition starting at 39° C., with increased decomposition and pressure build up at 60° C.

An experiment in a RC₁ calorimeter on the desired reaction revealed a 75% heat accumulation after all the N-benzyl protected β-carboline of Formula VII was added.

The reaction generated a moderate heat flow over about 3 hours and in the worst case the temperature would rise about 92° C., resulting in a MTSR of about 147° C. (MTSR refers to “maximum temperature of the synthetic reaction”). In the reaction of this example, the MTSR was determined by adding the starting temperature of 55° C. and the added heat production of about 92° C. (adiabatic temperature rise), to yield the MTSR of 147° C. The MTSR depends on the amount of unreacted starting materials at the moment of the cooling failure.

In the case of a cooling failure, a decomposition reaction will be triggered. Since the boiling point of the solvent DMF is higher then the MTSR, evaporative cooling cannot used to prevent the decomposition reaction.

To reduce the risk of decomposition involved in this reaction, a number of solvents were screened (Table 1):

| | without | |
|---|---|---|
| | Et₄NBr | with Et₄NBr |
| Dimethylformamide (DMF) | 80° C. | 75° C. |
| Acetonitrile (CH₃CN) | 90° C. | 70° C. |
| Dimethylacetamide (DMA) | 85° C. | 80° C. |
| Dimethylimidazolidinone (DMI) | 120° C. | 100° C. |

As shown in Table 1, the start temperature of the decomposition reaction when using KO₂-solvent mixtures (with or without Et₄NBr), is considerably higher in DMI as compared to DMF, CH₃CN and DMA. Although the degree of the decomposition remained the same, the probability for the decomposition to occur was lowered. In order to decrease the degree of possible decomposition some process development on the reaction in DMI was performed.

The excess of KO₂ was reduced from 8 to 6 equivalents, the amount of Et₄NBr was reduced from 8 to 1 equivalent and the reaction was performed more diluted i.e. 7 L/mole instead of 2.3 L/mole and at 50° C. As a result, the reaction time became longer and it now took 24 hours for complete conversion.

Using the adapted reaction conditions, additional RC₁ and Phi-tec experiments were performed.

The RC₁ experiment revealed that the reaction enthalpy was about 500 kJ/mole, but as the reaction was slow, the specific heat release was only 1.5 W/L and could easily be cooled in a production plant. However, there was about 90% thermal accumulation, so this is in fact a batch process and batch processes are not easy to control. In the Phi-tec calorimeter, the reaction or exothermic decomposition started at 52° C. with a total, Phi-tec corrected, adiabatic temperature rise of 192° C. At 70° C., the self heat rate was 0.56° C./min., with a maximum of 102° C./min. at 150° C. The adiabatic reaction temperature should not exceed 70° C., thus avoiding a runaway reaction (uncontrolled chemical reaction).

### Example 13

Oxidation of the N-benzyl protected β-carboline of Formula VII using mCPBA as the reagent mainly resulted in the N-oxide of the starting material. Accordingly, a protected β-carboline of Formula VII was reacted with mCPBA as shown in Scheme IV below. Suitable protecting groups for this reaction are selected from Boc or Cbz.

A 0.166 molar solution of the Boc protected (1R)-1-(2,3-dihydro-benzofuran-5-yl)-1,3,4,9-tetrahydro-β-carboline-2-carboxylic acid tert-butyl ester Compound 13 in DCM was reacted with a 0.166 molar solution of mCPBA (3 equivalents) in DCM at 20° C. over a one hour period.

The results showed that the reaction with mCPBA is dose controlled with respect to the dosing end point and the immediate heat release is dependent on the dosing time. Therefore, the reaction can be performed safely on a pilot plant scale. Moreover, the reflux of DCM prevented the reaction from reaching the MTSR.

After workup, the residue of the reaction contained 20-30% of the keto-lactam (3R)-3-(2,3-dihydro-benzofuran-5-yl)-2,7-dioxo-1,2,3,5,6,7-hexahydro-benzo[e][1,4]diazonine-4-carboxylic acid tert-butyl ester Compound 14 which on treatment with KOH was quantitatively converted to (3R)-3-(2,3-dihydro-benzofuran-5-yl)-9-oxo-1,3,4,9-tetrahydro-pyrrolo[3,4-b]quinoline-2-carboxylic acid tert-butyl ester Compound 15.

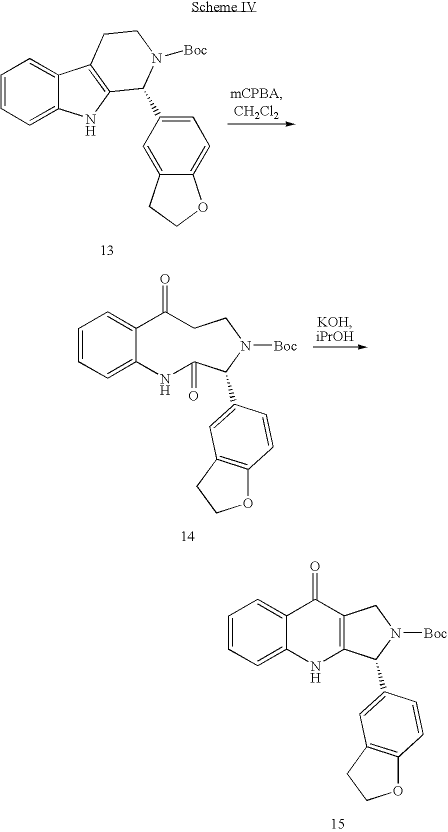

Attempts to isolate the keto-lactam 14 or compound 15 by crystallization failed and a chromatographic purification was required to obtain compound 15 with an acceptable purity.

### Example 14

Reacting Cbz protected (1R)-1-(2,3-dihydro-benzofuran-5-yl)-1,3,4,9-tetrahydro-β-carboline-2-carboxylic acid benzyl ester Compound 16 with mCPBA allowed isolation of the keto-lactam Compound 21 by crystallization from ethylacetate, as shown in Scheme VI. However, the formation of by-products was substantial, resulting in a keto-lactam yields of only about 20%.

### Example 15

Oxidation of 2,3-substituted indoles has been reported (Hino,T.; Yamaguchi,H.; Matsuki, K.; Nakano, K.; Sodeoka, M.; Nakagawa, M.; J. Chem. Soc. Perkin Trans. 1, 1983, 141-147) and a type of the formed intermediate was the hydroxy-imine 1,2,3,4-tetrahydro-carbazol-4a-ol Compound 19, as shown below in Scheme V.

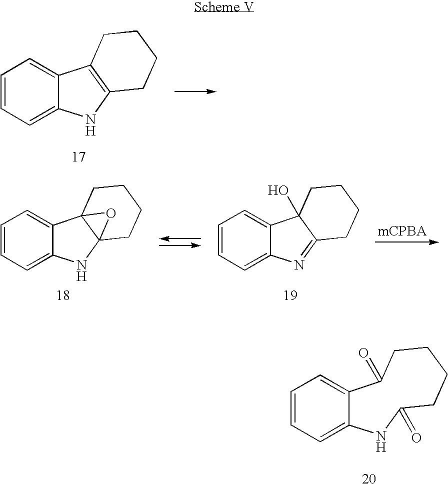

In the reported reaction, DMD can be used to epoxidize the 2,3,4,9-tetrahydro-1H-carbazole Compound 17 to provide a 2,3,4,5-tetrahydro-1H-carbazol [4a,9a-b]oxirane Compound 18 in equilibrium with the hydroxy-imine intermediate Compound 19. The intermediate can be converted to the keto-lactam 3,4,5,6-tetrahydro-1H-benzo[b]azonine-2,7-dione Compound 20 in good yields by further oxidation with mCPBA.

Alternatively, the epoxide Compound 18 can be formed in situ from acetone and OXONE® at controlled pH (as described in Adam, W.; Chan, Y.-T.; Cremer, D.; Gauss, J.; Scheutzow, D.; Schlinder, M.; J. Org. Chem. 1987, 52, 2800-2803; (b) Murray, R. W.; Jeyaraman, R. J. Org. Chem. 1985, 50, 2847-2853).

A Phi-Tec experiment on in situ generated DMD-solutions in a variety of solvents such as water/ethylacetate/acetone and NaHCO₃ showed that these solutions were thermally stable up to 70° C.

### Example 16

A solution of the Cbz protected (1R)-1-(2,3-dihydro-benzofuran-5-yl)-1,3,4,9-tetrahydro-β-carboline-2-carboxylic acid benzyl ester Compound 16 (referred to in EXAMPLE 14) in ethylacetate/acetone was reacted with OXONE® and mCPBA, as shown below in Scheme VI.

Compound 16 was oxidized using OXONE® and mCPBA to provide the keto-lactam (3R)-3-(2,3-dihydro-benzofuran-5-yl)-2,7-dioxo-1,2,3,5,6,7-hexahydro-benzo[e][1,4]diazonine-4-carboxylic acid benzyl ester Compound 21. The keto-lactam Compound 21 was isolated by crystallization from ethylacetate with an overall yield of 36% on a 46 mole scale.

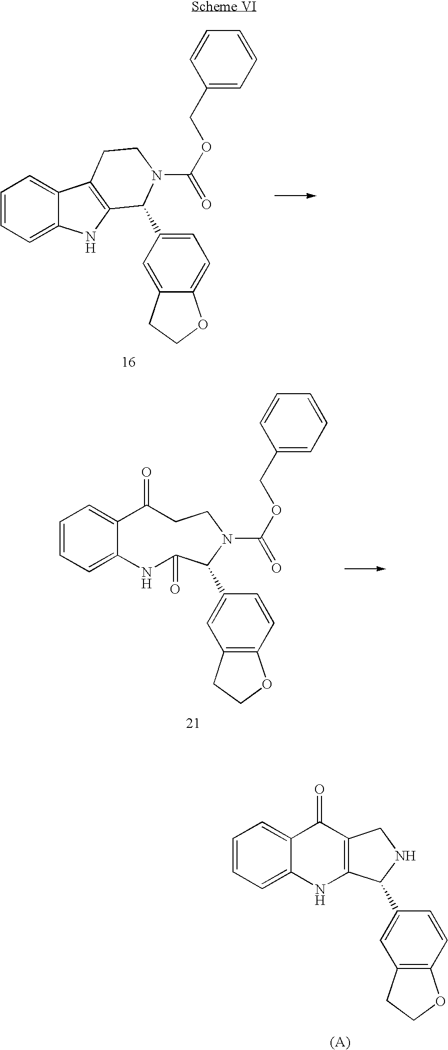

Though the yield of this reaction was nearly doubled it still seemed that the mCPBA sequence was responsible for the still relatively low yield.

After the DMD-oxidation step the reaction mixture contained up to 60-70% (HPLC area percent) of the hydroxy-imine (identification by LC-MS) and again most of the impurities started to form upon addition of mCPBA.

Starting materials, reagents and solvents were obtained from commercial suppliers and were used without any further purification.

¹H NMR spectra were recorded at 400 MHz on a Brucker Avance-400 instrument.

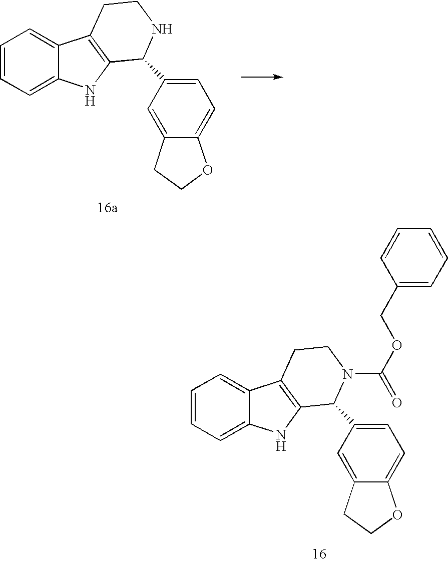

A 250 mL vessel was charged successively with (1R)-1-(2,3-dihydro-benzofuran-5-5yl)-2,3,4,9-tetrahydro-1H-β-carboline Compound 16a (5.22 g, 0.018 mole), ethylacetate (150 mL) and triethylamine (2.1 g, 0.0208 mole). The mixture was heated to a temperature of from about 40° C. to about 45° C. Then the heating was stopped and benzylchloroformate (3.55 gr., 0.0208 mole) was added dropwise. The addition was slightly exothermic and the temperature rose from 42 to 51° C.

The mixture was stirred for another 2 hours at a temperature of from about 51° C. to about 42° C. Water (18 mL) was added and the layers were separated while keeping the temperature at about 35° C. to about 40° C. To the organic layer acetone (107 mL) was added and the mixture was cooled to a temperature of from about 20° C. to about 25° C. Subsequently, water (36 mL) and NaHCO₃ (4.82 g, 0.057 mole) were added and the mixture was further cooled to a temperature of from about 0° C. to about 5° C. to provide the protected (1R)-1-(2,3-dihydro-benzofuran-5-yl)-1,3,4,9-tetrahydro-β-carboline-2-carboxylic acid benzyl ester Compound 16, which was not isolated in this step.

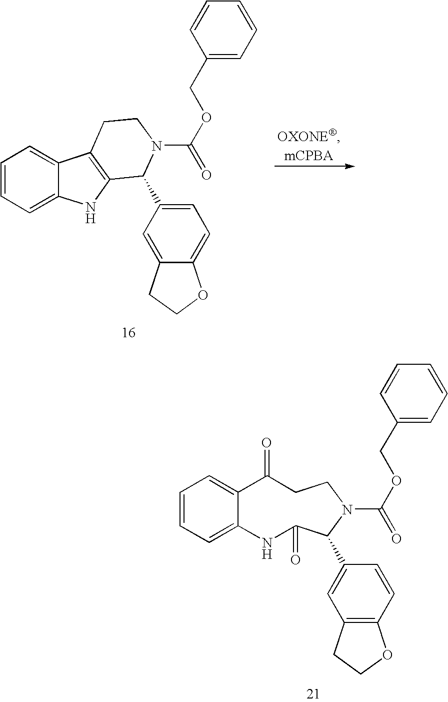

A solution of OXONE® (15.5 g, 0.025 mole) in water (60 mL) was added to the mixture of Compound 16 by means of a dosing pump over a period of about 2 hours at a temperature of from about 0° C. to about 5° C. After the addition was complete, the mixture was stirred for another 2 hours at a temperature of from about 0° C. to about 5° C. A solution of mCPBA (71%, 5.34 g, 0.217 mole) in dichloromethane (62 mL) was added over a period of about 15 minutes at a temperature of from about 0° C. to about 5° C. The mixture was stirred for another 1.5 hour at a temperature of from about 0° C. to about 5° C. and then washed successively with water (76 mL) and with a solution of NaHCO₃ (6 g) in water (76 mL).

After checking for peroxides, the organic layer was evaporated to near dryness and the resulting oily residue was crystallized from ethyl acetate (40 mL). After stirring for 18 hours at a temperature of from about 10° C. to about 25° C., the resulting precipitate was filtered, washed with ethyl acetate (2 mL) and dried in vacuo at 50° C. for 20 hours yielding (3 g, 36.5%) of (3R)-3-(2,3-dihydro-benzofuran-5-yl)-2,7-dioxo-1,2,3,5,6,7-hexahydro-benzo[e][1,4]diazonine-4-carboxylic acid benzyl ester Compound 21.

¹H NMR (400 MHz, DMSO-D6) showed a mixture of rotamers: δ ppm 2.75-2.90 (m, 2H); 3.08-3.19 (m, 3H); 4.45-4.53 (o, 3H); 5.01-5.24 (4×d (o), 2H); 5.93-5.96 (2×s, 1H); 6.67-6.72 (2×d (o), 1H); 6.84-6.90 (2×d (o)n 1H); 7.01-7.03 (2×s, 1H); 7.19-7.67 (o.m. 9H); 10.84-10.86 (2×s, 1H).

Other probable rotamers were observed: 3.38-4.28 (m); 4.60-4.77 (2 d); 5.54 (s), 5.61 (s); 9.68 (s), 9.73 (s).

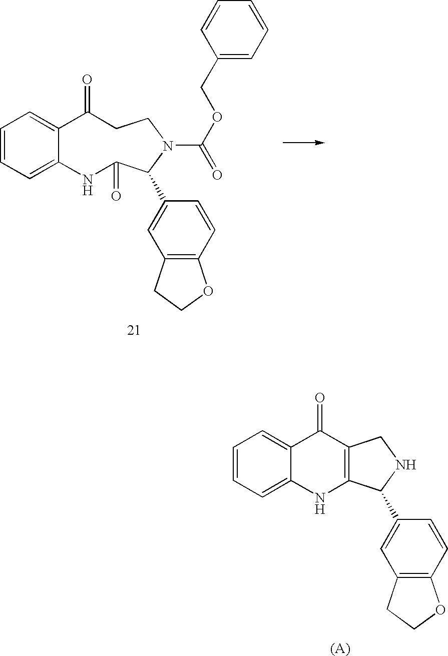

A 1 Liter flask was charged successively with Compound 21 (45.6 g, 0.1 mole) and ethanol (400 mL). To the resulting suspension was added a 50% aqueous solution of NaOH (5.8 mL, 0.11 mole) and the mixture was stirred for 1 hour at a temperature of from about 20° C. to about 25° C. Concentrated HCl (9.9 ml, 0.11 mole) was added dropwise and after stirring for another 30 minutes, the precipitated salts were filtered and washed with ethanol (100 mL). The combined filtrates were transferred into a hydrogenation vessel and concentrated HCl (9 mL, 0.1 mole) was added. The solution was flushed with nitrogen, then palladium on charcoal (in a mixture with about 5% to about 50% water, 5 g) was added and subsequently hydrogenated at a temperature of from about 25° C. to about 30° C.

When deprotection was complete, the mixture was filtered. NH₄OH (10 mL, 0.13 mole) was added to the filtrate followed by a dropwise addition of water (550 mL). The solution was then seeded with (3R)-3-(2,3-dihydro-benzofuran-5-yl)-1,2,3,4-tetrahydro-pyrrolo[3,4-b]quinolin-9-one compound of formula (A) (0.1 g). When crystallization had started another portion of water (550 mL) was added dropwise over a period of about 1 hour. The mixture was stirred for another 18 hours, after which the precipitate was filtered and washed with water (50 mL), then dried at 50° C. for 18 hours to provide the compound of formula (A) (22.7 g, 75%).

¹H NMR (400 MHz, DMSO-D6) δ ppm 3.13 (t, J=8.69 Hz, 2H); 4.02 (dd, J=12.97, 1.38 Hz, 1H); 4.18 (dd, J=12.97, 2.90 Hz, 1H); 4.50 (t, J=9.06 Hz, 2H); 5.37 (dd, J=2.77, 1.76 Hz, 1H); 6.73 (d, J=8.31 Hz, 1H); 7.07 (dd, J=8.31, 1.76 Hz, 1H); 7.17 (d, J=1.26 Hz, 1H); 7.26-7.31 (m, J=8.06, 5.79, 2.01 Hz, 1H); 7.53-7.57 (m, 1H); 7.56-7.60 (m, 1H); 8.11-8.15 (m, 1H); 11.58 (s, 1H).

What is claimed is:

1. A process for preparing a compound of formula I 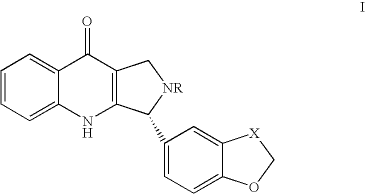
wherein X is selected from the group consisting of —CH₂—, NH, O and S;
R is selected from H, C₁₋₁₀alkyl, C₁₋₁₀alkyl substituted with C₁₋₆alkyloxy, or C₁₋₁₀alkyl substituted with halo, aryl, heteroaryl, heterocycle, C(O)-C₁₋₁₀alkyl, C(O)-aryl, C(O)O-C₁₋₁₀alkyl or C(O)O-aryl or a salt thereof, and
wherein N-acetyl-D-leucine is used.

2. A process according to claim 1, wherein N-acetyl-D-leucine is used as a resolving agent.

3. A process according to claim 1, comprising the process step of resolving an intermediate of formula III, wherein X is selected from the group consisting of CH₂, NH, O or S; with N-acetyl-D-leucine:

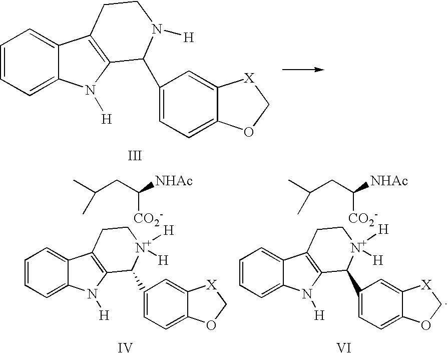

4. A process for resolving an intermediate of formula III, wherein X is selected from the group consisting of CH₂, NH, O or S, with N-acetyl-D-leucine:

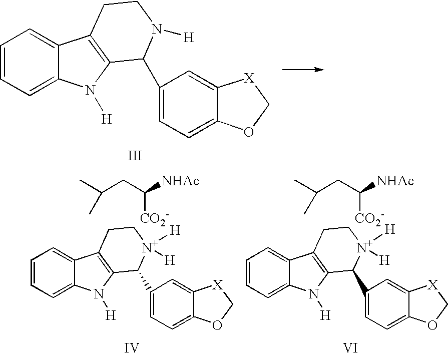

5. A process for preparing a compound of formula I, wherein X is selected from the group consisting of CH₂, NH, O or S, comprising the steps of:
a) preparing an intermediate of formula III by reacting tryptamine with an aldehyde of formula II: 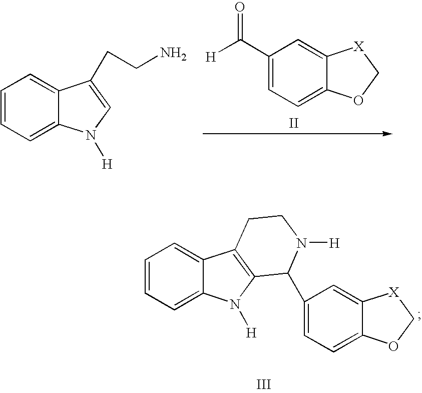
b) preparing an enantiomerically enriched intermediate of formula IV, by reacting the intermediate of formula m with N-acetyl-D-leucine: 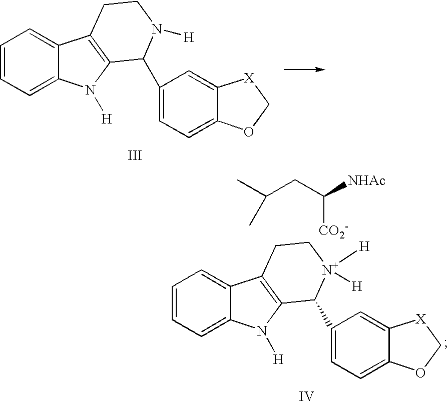
c) preparing an intermediate of formula VII by protecting an intermediate of formula IV: 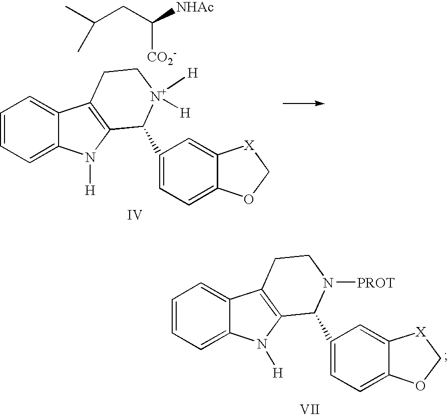
d) transforming an intermediate of formula VII into an intermediate of formula VIII: 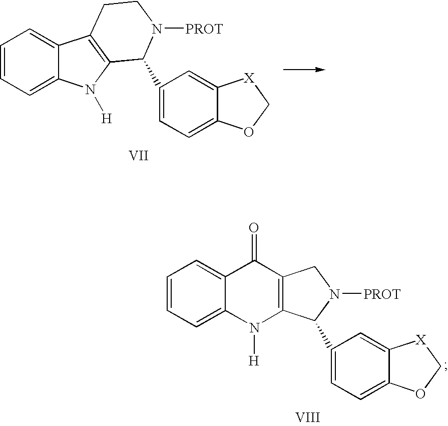
e) deprotecting an intermediate of formula VIII, thus obtaining a compound of formula I, wherein R is hydrogen (being represented by formula I-H): 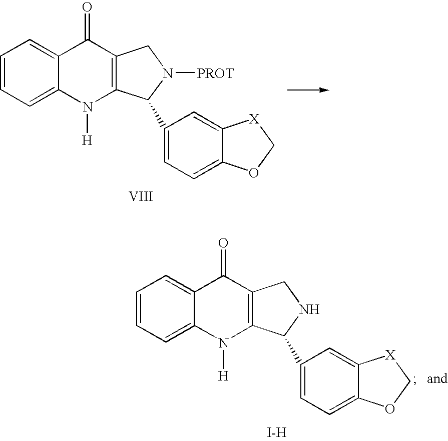
f) converting the compound of formula I-H into a compound of formula I.

6. A process for preparing a compound of formula I, wherein R is hydrogen and X is CH₂, comprising the steps of
a) preparing an intermediate of formula III-C by reacting tryptamine with the aldehyde of formula II-C: 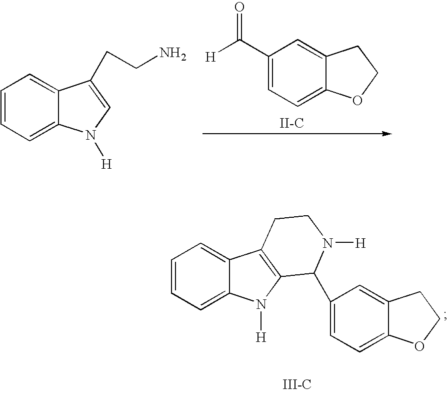
b) preparing an enantiomerically enriched intermediate of formula IV-C by reacting the intermediate of formula III-C with N-acetyl-D-leucine: 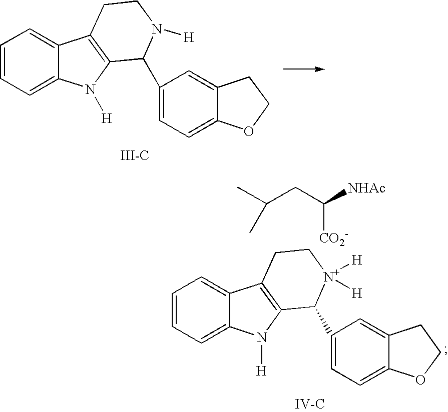
c) preparing an intermediate of formula VII-C by protecting the intermediate of formula IV-C: 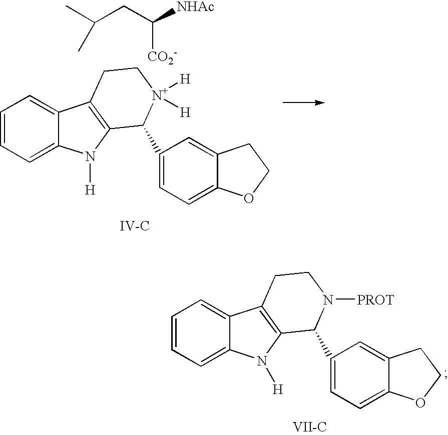
d) transforming an intermediate of formula VII-C into an intermediate of formula VIII-C: 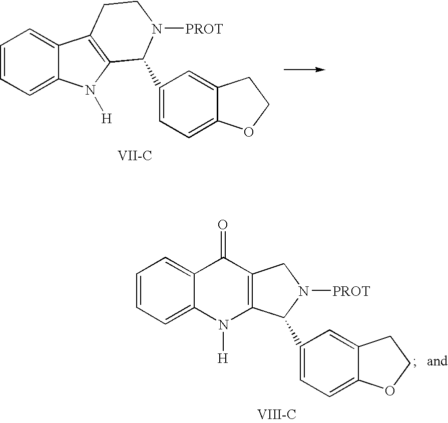
e) deprotecting the intermediate of formula VII-C, thus obtaining a compound of formula I-C:

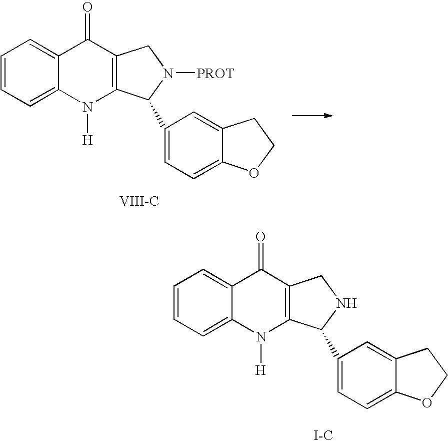

7. A process for epimerization of a (S)-enantiomer enriched (1S)-1-(2,3-dihydrobenzofuran-5-yl)-2,3,4,9-tetrahydro-1H-β-carboline mixture to a near racemic (±)-1-(2,3-dihydrobenzofuran-5-yl)-2,3,4,9-tetrahydro-1H-β-carboline mixture.

8. A process as claimed in claim 7, wherein the chemical yield of near racemic (±)-1-(2,3-dihydrobenzofuran-5-yl)-2,3,4,9-tetrahydro-1H-β-carboline mixture is above 90%.

9. A process as claimed in claim 7, wherein trifluoroacetic acid is used as a catalyst.

10. A process as claimed in claim 1, wherein one of the steps is epimerization of a (S)-enantiomer enriched (1S)-1-(2,3-dihydrobenzofuran-5-yl)-2,3,4,9-tetrahydro-1H-β-carboline mixture.

11. A process for preparing a compound of formula 12: 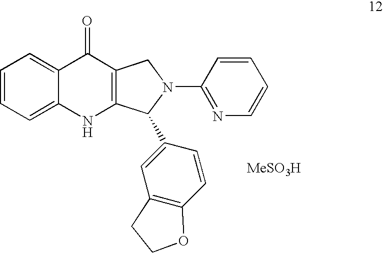
comprising the steps of
a) reacting a compound of formula (A): 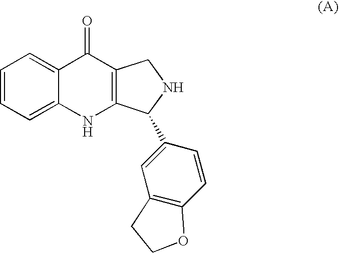
with 2-bromo-pyridine in the presence of a palladium catalyst and a BINAP ligand in THF to provide a compound of formula 11: 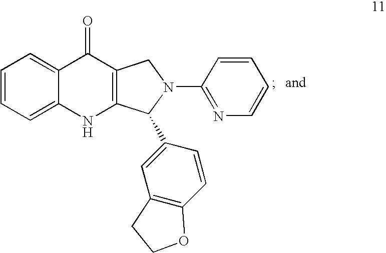
b) reacting the compound of formula 11 with methanesulfonic acid.

12. A process for preparing a compound of formula (A): 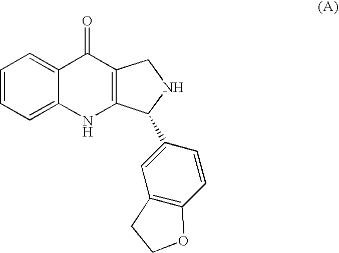
comprising the steps of
a) reacting a compound of formula 16: 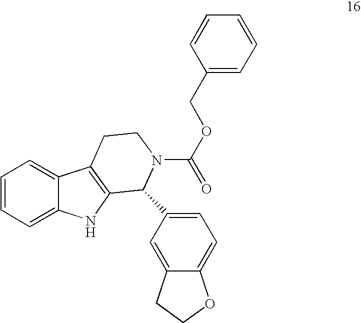
with potassium peroxy monosulfate and 3-chloroperoxybenzoic acid to provide a compound of formula 21: 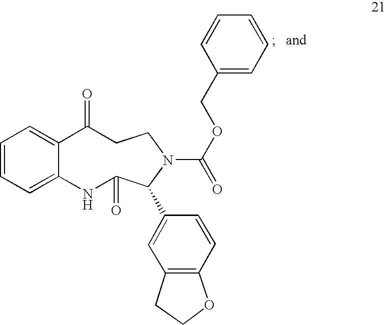
b) reacting the compound of formula 21 with an aqueous base to provide an intermediate, followed by deprotection of the intermediate.
